(19) United States
(12) Patent Application Publication
(10) Pub. No.: US 2010/0303935 A1
(43) Pub. Date: Dec. 2, 2010

(54) Medicinal Composition

(76) Inventor: Meryl J. Squires, Barrington Hills, IL (US)

Correspondence Address: Tolpin & Partners, PC, 11 S. LaSalle Street, Suite 2900, Chicago, IL, 60603, US

(21) Appl. No.: 12/474,694

(22) Filed: May 29, 2009

(51) Int. Cl. A61K 36/00; A61K 36/28; A61K 36/75; A61K 36/30; A61K 36/38; A61K 36/886; A61K 36/22; A61K 36/328; A61K 36/71; A61K 36/81; A61K 36/61; A61P 29/00

(52) U.S. Cl. 424/726; 424/725; 424/737; 424/730; 424/744; 424/760; 424/742

(57) ABSTRACT

A medicinal composition comprising herbal extracts of herbaceous botanicals that can help provide relief of various ailments. The medicinal composition can comprise a topical analgesic with therapeutic benefits for use in humans, equine, bovine, canine, feline, porcine, or other animals or birds for treatment, healing or relieving symptoms resulting from injured ligaments, tendons, muscles, bones, hematomas, nerves, or sports injuries or for epidermal or dermal treatment or relief of conditions or symptoms caused by arthritis, neuralgia, or pain. The herbaceous botanicals of the preferred herbal extracts of herbaceous botanicals in this application can include: Bellis Perennis (Daisy), Ruta Graveolens (Rue), Comfrey, Elder, Hops, Echinacea, Hypericum Perforatum (St. John's Wart), Aloe Vera, Mistletoe, Rhus toxicodendron (Poison Ivy), Eucalyptus, Commiphora myrrha, Goldenseal, and Cayenne. Furthermore, the medicinal composition can include Benzalkonium halide, such as Benzalkonium halide chloride, as well as at least one compound comprising Menthol or a diluent comprising distilled water and/or Isopropyl Alcohol.

### BACKGROUND OF THE INVENTION

This invention pertains to a composition and, more particularly, to a medicinal composition for help treating various ailments.

Many people as well as equine, bovine, canine, feline, porcine, or other animals or birds are in need of treatment and healing of ligaments, tendons, muscles, bones, hematomas, nerves, or pain. Furthermore, many people are also in need of treatment and healing of arthritis, neuralgia, minor wounds, bone bruises, tendon injuries, sport injuries or other injuries.

Over the years, various products and treatments have been developed or suggested for curing, treating or relieving one or more of the preceding ailments. These previous products and treatment have met with varying degrees of success.

It is, therefore, desirable to provide an improved medicinal composition and treatment which helps alleviate most, if not all, of the preceding problems.

### BRIEF SUMMARY OF THE INVENTION

An improved medical (medicinal) composition is provided with medicinal herbal extracts of herbaceous botanicals to help provide relief of various ailments and/or their symptoms. Advantageously, the medicinal composition is effective, economical, and easy to use. The composition can comprise a topical analgesic, counter-irritant, pharmaceutical product, medicinal preparation, or medicine. The medicinal composition can also comprise a veterinary medicine or veterinary medicinal product to help treat or relieve various ailments in dogs, cats, birds, horses, cows, sheep, swine (pigs or hogs), or other farm animals, rodents, or animals in zoos. In some circumstances, it may be useful to use the novel medicinal composition therapeutically, externally, locally, internally, and/or systemically.

The topical analgesic can have therapeutic benefits for use in humans, equine, bovine, canine, feline, porcine, or other animals or birds for treatment and healing of ligaments, tendons, muscles, bones, hematomas, nerves, or sports injuries, or for epidermal or dermal treatment of arthritis, neuralgia, or pain; as well as for topical treatment for minor wounds and injuries.

Significantly, the medicinal composition can comprise a first plant group of 2-5 different primary plants consisting of extracts or portions of primary herbaceous botanicals of the genus Bellis Perennis (Daisy), Ruta Graveolens (Rue), Comfrey, Elder, and/or Hops. The medicinal composition can also comprise a second plant group of 1-4 different secondary plants of extracts or portions of secondary herbaceous botanicals of the genus Echinacea, Hypericum Perforatum (St. John's Wart), Aloe, and/or Mistletoe. The Aloe can comprise extracts or portions of an Aloe plant consisting of Aloe Barbadensis, Aloe Capensis, and/or Aloe Vera. The medicinal composition can further comprise a third plant group of tertiary plants consisting of extracts or portions of 1-6 different tertiary herbaceous botanicals of the genus Rhus toxicodendron (Poison Ivy), Commiphora myrrha, Goldenseal, Cayenne, Eucalyptus, and/or Camphor. Significantly, the medical composition desirably excludes undesired Myrtle and Centaurea and their extracts, which can inhibit or retard the unexpected advantageous results of the novel medicinal composition. Furthermore, the medicinal composition can include Benzalkonium halide such as Benzalkonium chloride, Benzalkonium bromide, or Benzalkonium fluoride, as well as at least one compound comprising Menthol or a diluent comprising distilled water and/or Isopropyl Alcohol. Desirably, the herbaceous botanicals cooperate with each other in the medicinal composition, as well as with any Benzalkonium halide, Menthol or a diluent comprising distilled water and/or Isopropyl Alcohol, to help treat ailments or diseases, or relieve various conditions or symptoms associated with different ailments or diseases in persons and animals.

In the preferred form, the medicinal composition comprises by weight based upon the total weight of the composition:
primary herbal extracts of at least a portion of five (5) different primary plants consisting of the following primary herbaceous botanicals
   from about 0.01% to about 90% Bellis Perennis (Daisy);
   from about 0.003% to about 92% Ruta Graveolens (Rue);
   from about 0.001% to about 92% Comfrey,
   from about 0.003% to about 94% Elder;
   from about 0.001% to about 88% Hops;
secondary herbal extracts of at least a portion of three (3) different secondary plants consisting of the following secondary herbaceous botanicals
   from about 0.01% to about 90% Echinacea purpurea;
   from about 0.005% to about 98% Hypericum Perforatum (St. John's Wart);
   from about 0.002% to about 95% Aloe;
   from about 0% to about 84% Mistletoe;
tertiary herbal extracts of at least a portion of six (6) different tertiary plants consisting of the following tertiary herbaceous botanicals
   from about 0.001% to about 98% Rhus toxicodendron (Poison Ivy);
   from about 0.004% to about 86% Commiphora myrrha;
   from about 0.002% to about 83% Goldenseal;
   from about 0.5% to about 75% Cayenne;
   from about 0.1% to about 10% Eucalyptus;
   from about 0.1% to about 10% Camphor;
from about 0% to about 85% Menthol;
from about 0.03% to about 27% Benzalkonium halide; and
a diluent comprising from about 0.02% to about 98% Isopropyl Alcohol and/or from about 40% to about 60% water.

A more detailed explanation of the invention is provided in the following description and appended claims.

### DETAILED DESCRIPTION OF THE INVENTION

The following is a detailed description of the preferred embodiments and best modes for practicing the invention.

An improved medicinal composition is provided with herbal extracts of herbaceous botanicals to help provide deep relief and/or help quickly resolve various ailments or conditions associated with those ailments. The medicinal composition comprises by weight based upon the total weight of the medicinal composition:
primary herbal extracts of at least a portion of five (5) different primary plants consisting of the following primary herbaceous botanicals
   from about 0.01% to about 90% Bellis Perennis (Daisy);
   from about 0.003% to about 92% Ruta Graveolens (Rue);
   from about 0.001% to about 92% Comfrey,
   from about 0.003% to about 94% Elder;
   from about 0.001% to about 88% Hops;
secondary herbal extracts of at least a portion of three (3) different secondary plants consisting of the following secondary herbaceous botanicals
   from about 0.01% to about 90% Echinacea;
   from about 0.005% to about 98% Hypericum Perforatum (St. John's Wart);
from about 0.002% to about 95% Aloe (Aloe Barbadensis, Aloe Capensis, and/or preferably Aloe Vera);
   from about 0% to about 84% Mistletoe;
tertiary herbal extracts of at least a portion of six (6) different tertiary plants consisting of the following tertiary herbaceous botanicals
   from about 0.001% to about 98% Rhus toxicodendron (Poison Ivy);
   from about 0.004% to about 86% Commiphora myrrha;
   from about 0.002% to about 83% Goldenseal; and
   from about 0.5% to about 75% Cayenne;
   from about 0.1% to about 10% Eucalyptus;
   from about 0.1% to about 10% Camphor;
from about 0% to about 85% Menthol;
from about 0.03% to about 27% Benzalkonium halide (Benzalkonium bromide, Benzalkonium fluoride and/or most preferably Benzalkonium chloride);
from 0.001% to about 85% Menthol; and
a diluent comprising from about 0.02% to about 98% isopropyl alcohol and/or from about 40% to about 60% water.

Preferably, the medicinal composition comprises by weight based upon the total weight of the medicinal composition:
primary herbal extracts of at least a portion of five (5) different primary plants consisting of the following primary herbaceous botanicals
   from about 4% to about 8% Bellis Perennis (Daisy);
   from about 4% to about 8% Ruta Graveolens (Rue);
   from about 4% to about 8% Comfrey;
   from about 4% to about 8% Elder;
   from about 4% to about 8% Hops;
secondary herbal extracts of at least a portion of three (3) different secondary plants consisting of the following secondary herbaceous botanicals
   from about 2% to about 4% Echinacea purpurea;
   from about 2% to about 4% Hypericum Perforatum (St. John's Wart);
   from about 2% to about 4% Aloe Vera;
   from about 0%, preferably from about 2%, to about 4% Mistletoe;
tertiary herbal extracts of at least a portion of six (6) different tertiary plants consisting of the following tertiary herbaceous botanicals
   from about 0.5% to about 1.5% Rhus toxicodendron (Poison Ivy);
   from about 0.5% to about 1.5% Commiphora myrrha;
   from about 0.5% to about 1.5% Goldenseal; and
   from about 0.5% to about 1.5% Cayenne;
   from about 1% to about 4% Eucalyptus;
   from about 1% to about 4% Camphor;
from about 2% to about 4% Menthol;
from about 0.05% to about 0.2% Benzalkonium chloride;
a diluent comprising from about 60% to about 80% Isopropyl Alcohol and/or from about 40% to about 60% distilled water.

The herbal extracts can cooperate with the Menthol, Isopropyl Alcohol, Benzalkonium Chloride and/or distilled water in the medicinal composition to help treat or relieve ailments, diseases, and infections or conditions associated therewith.

Most preferably, the medicinal composition comprises by weight based upon the total weight of the medicinal composition:
primary herbal extracts of at least a portion of five (5) different primary plants consisting of the following primary herbaceous botanicals
   about 6% Bellis Perennis (Daisy);
   about 6% Ruta Graveolens (Rue);
   about 6% Comfrey;
   about 6% Elder;
   about 6% Hops;
secondary herbal extracts of at least a portion of three (3) different secondary plants consisting of the following secondary herbaceous botanicals
   about 3% Echinacea purpurea;
   about 3% Hypericum Perforatum (St. John's Wart);
   about 3% Aloe Vera;
   about 0% to about 3% Mistletoe;
tertiary herbal extracts of at least a portion of six (6) different tertiary plants consisting of the following tertiary herbaceous botanicals
   about 1% Rhus toxicodendron (Poison Ivy);
   about 1% Commiphora myrrha;
   about 1% Goldenseal;
   about 1% Cayenne;
   about 2.5% Eucalyptus;
   about 2.5% Camphor;
about 0% to about 3% Menthol;
about 0.13% Benzalkonium chloride;
a diluent comprising about 70% Isopropyl Alcohol and/or from about 51% to about 52% distilled water.

### Herbal Extracts

Raw, untreated, unprocessed, non-isolated plants comprising herbaceous botanical are often undesirable and/or ineffective to treat various ailments and cure various diseases. Treated and/or processed plants comprising herbaceous botanicals, when, appropriately filtered and/or isolated into constituents comprise medicinal herbal extracts which provide phytochemicals that can be effective to treat, alleviate, or relieve various ailments, diseases or infections, or conditions associate with the preceding. Significantly, it appears that many of the isolated constituents that comprise herbal extracts of herbaceous botanicals provide antimicrobial isolates, botanical extracts and microbe inhibiters which have or exhibit antimicrobial activity that appear to be effective in treating or relieving conditions associated with various ailments, diseases, or infections.

As discussed previously, the preferred herbal extracts of herbaceous botanicals in this application are: Bellis Perennis (Daisy), Ruta Graveolens (Rue), Comfrey, Elder, Hops, Echinacea, Hypericum Perforatum (St. John's Wart), Aloe Vera, Mistletoe, Rhus toxicodendron (Poison Ivy), Eucalyptus, Commiphora myrrha, Goldenseal, and Cayenne.

Significantly, the preferred medical composition excludes undesired Myrtle and/or Centaurea and their extracts, which can inhibit or retard the unexpected advantageous results of the novel medicinal composition.

### Bellis Perennis

### Daisy

Bellis Perennis (Daisy) has a botanical name of Bellis perennis (LINN) and is from the family N.O. Compositae. Daisy is also known as Bruisewort. (Scotch) Bairnwort. (Welsh), Llygad y Dydd (Eye of the Day), Great Ox-eye. Goldens. Marguerite. Moon Daisy. Horse Gowan. Maudlin Daisy. Field Daisy. Dun Daisy. Butter Daisy. Horse Daisy. Maudlinwort. White. Weed. Leucanthemum vulgare. (Scotch)-Gowan. The parts of Daisy that are used as an herb include the flower and roots and sometimes the entire plant.

Daisy can be found throughout Europe and Russian Asia. The genus derives its name from the Greek words chrisos (golden) and anthos (flower). The specific name of the ox-Eye signifies ‘white flower,’ being like the generic name, and is Greek in origin. The old northern name for the Daisy is Baldur's Brow, and this, with many other species of Chrysanthemum became dedicated to St. John.

Daisy generally grows from 1 to 2 feet high. The root of Daisy is perennial and somewhat creeping; the stems, hard and wiry, furrowed and only very slightly branched. The leaves of Daisy are small and coarsely toothed; those near the root are somewhat rounder in form than those on the stem, and are on long stalks, those on the stem are oblong and stalkless. By the middle of May, the familiar yellow centered white flower-heads commence to bloom, and are at their best till about the close of June, though isolated specimens may be met with throughout the summer, especially where undisturbed by the cutting of the hay, as on railway bed, where the plant flourishes well. Beneath each flower-head is a ring of green sheathing bracts, the involucres. These not only protect and support the bloom, but helps prevent insects trying to bite their way to the honey from below. They, as well as the rest of the plant, are permeated with an acrid juice that can be obnoxious to insects.

Daisy can be an antispasmodic diuretic, tonic. Ox-Eye Daisy can be used to treat whooping-cough, asthma and nervous excitability. As a tonic, Daisy can be used to treat night sweats. The flowers of Daisy are balsamic and make a useful infusion for relieving chronic coughs and for bronchial catarrhs. Externally, Daisy can be used as a lotion for wounds, bruises, ulcers and some coetaneous diseases.

The composition of Daisy comprises: saponins, tannin, essential oil, flavones, bitter principle, and mucilage.

### Ruta Graveolens

### Rue

Ruta Graveolens (Rue) has a botanical name of Ruta graveolens (LINN) in the family N.O. Rutaceae and is sometime known as Garden Rue, Herb of Grace, or Herbygrass. Rue is a hardy, evergreen, somewhat shrubby plant and is a native of Southern Europe. The stem is woody in the lower part, the leaves are alternate, bluish-green, bi- or tripinnate, emit a powerful, disagreeable odor and have a bitter, acrid and nauseous taste. The greenish-yellow flowers are in terminal panicles, blossoming from June to September. In England, Rue is one of the oldest garden plants, and is cultivated for its use medicinally, having, together with other herbs, been introduced by the Romans, but it is rarely found in a wild state. This wild form is even more vehement in smell than the garden Rue. The first flower that opens has usually ten stamens, the others eight only. Rue is an herb and is preferably collected before the flowers open in the summer and dried in the shade.

Rue grows almost anywhere, but thrives best in a partially sheltered and dry situation. Propagation may be effected: (1) by seeds, sown outside, broadcast, in spring, raked in and the beds kept free from weeds, the seedlings, when about 2 inches high, being transplanted into fresh beds, allowing about 18 inches each way, as the plants become bushy; (2) by cuttings, taken in spring and inserted for a time, until well rooted, in a shady border; (3) by rooted slips, also taken in spring. Every slip or cutting of the young wood can readily grow, and this is the most expeditious way of raising a stock. Rue can live much longer and can be less likely to be injured by frost in winter when grown in a poor, dry, rubbishy soil.

The name Ruta is derived from the Greek reuo (to set free), because the herb is so efficacious in various diseases. It was much used by the ancient Greeks. Hippocrates specially commended it, and it constituted a chief ingredient of the famous antidote to poison used by Mithridates. The Greeks regarded it as an antimagical herb, because it served to remedy the nervous indigestion they suffered when eating before strangers, which they attributed to witchcraft. Piperno, a Neapolitan physician, in 1625, commended Rue as a specific against epilepsy and vertigo, and for the former malady, at one time, some of this herb used to be suspended round the neck of the sufferer. Rue is used by some people to make the sight both sharp and clear, especially when the vision had become dim through over-exertion of the eyes.

The whole of Rue can be used as an herb and can comprise fresh and/or dried herb. The tops of the young shoots of Rue contain the greatest virtues of any part of the plant. The shoots are gathered before the plant flowers. Volatile oil is contained in glands of Rue and is distributed over the whole plant and contains caprinic, plagonic, caprylic and oenanthylic acids—also a yellow crystalline body, called rutin. The oil of Rue can be distilled from the fresh herb. Water serves to extract the virtues of the plant better than spirits of wine. Decoctions and infusions are usually made from the fresh plant. The oil of Rue may be given in a dose of from 1 to 5 drops.

The composition of Rue comprises: volatile oil, 2-undecanone (50-90%), 2-haptanol, 2-nonanol, 2-nonanone, limonene, pinene, anisic acid, phenol, guiacol and others: flavonoids such as quercitin and rutin; coumarins: bergapten, daphnoretin, isoimperatorin, naphthoherniarin, psoralen, pangelin, rutamarin, rutarin, scopoletin and umbelliferone; alkaloids: arborinine, [[gamma]]-fagarine, graveoline, graveolinine, kokusaginine, rutacridine; and lignans, in the root; savinin and helioxanthin.

Rue can be strongly stimulating and antispasmodic. Rue can be used in the form of a warm infusion as an emmenagogue. In excessive doses, Rue is an acro-narcotic poison, and on account of its emetic tendencies should not be administered immediately after eating. Rue can be a useful medicine in hysterical affections, in coughs, croupy affections, colic and flatulence, being a mild stomachic. Externally, Rue is an active irritant, being employed as a rubefacient. If Rue is bruised and applied, the leaves of Rue can sometimes ease the severe pain of sciatica. The fresh leaves of Rue applied to the temples can sometimes relieve a headache. Compresses saturated with a strong decoction of Rue, when applied to the chest, can be used beneficially for chronic bronchitis.

### Comfrey

Comfrey (Symphytum officinale L.) is a perennial herb of the family Boraginaceae with a black, turnip-like root and large, hairy broad leaves that have small bell-shaped white, cream, purple or pink flowers. Comfrey is native to Europe, growing in damp, grassy places, and is widespread throughout the British Isles on river banks and ditches. Comfrey is derived from the Latin word for “grow together” and is also known as “knitbone”.

Comfrey species include: Bocking 14″ cultivar of Russian Comfrey (Symphytum×uplandicum); Symphytum asperum, Prickly Comfrey, Rough Comfrey (Symphytum asperrimum); Symphytum bulbosum, Bulbous Comfrey; Symphytum caucasicum, Caucasian Comfrey; Symphytum grandiflorum, Creeping Comfrey (Symphytum ibericum); Symphytum orientale, White Comfrey; Symphytum tauricum, Crimean Comfrey; Symphytum tuberosum, Tuberous Comfrey; Healing Herb, Blackwort, Bruisewort, Wallwort, Gum Plant. (Symphytum. asperum×officinale, synonym: S. peregrinum). Wild or common Comfrey, Symphytum officinale L., is native to England and extends throughout most of Europe into Central Asia and Western Siberia. Prickly or rough Comfrey [S. asperum Lepechin (Symphytum. asperrimum Donn)], named for its bristly or hairy leaves, was brought to Great Britain from Russia about 1800. Quaker, Russian, or blue Comfrey [Symphytum×uplandicum Nyman (Symphytum. peregrinum Lebed.)] originated as a natural hybrid of Symphytum. officinale L. and Symphytum. asperum Lepechin.

The composition of Comfrey includes: allantoin, mucilage (fructans), intermedine, acetylintermedine, lycopsamine, acetyllcopsamine, symphytine, echimedine, steroidal saponins, sterols (sitosterol), tannins, rosmarinic acid, pyrrolizidine alkaloids, startch, triterpens (isobauerenol), lithospemic acid, asparagine, amino acids, inulin, vitamin B12, and proteins. Dried Comfrey leaves contain 0.1% to 1.6% by weight allantoin, while dried Comfrey roots contain 0.4% to 1.5% by weight allantoin. Fresh Comfrey leaves are 85% by weight water and contain less than 0.2% by weight allantoin.

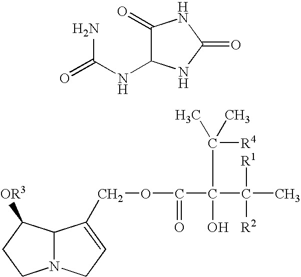

| R | R¹ | R² | R³ | R⁴ |
|---|---|---|---|---|
| Intermedine | OH | H | H | H |
| Acetylintermedine | OH | H | Acetyl | H |
| Lycopsamine | H | OH | H | H |
| Acetyllycopsamine | H | OH | Acetyl | H |
| Symphytine | H | OH | Tigloyl | H |
| Echimidine | H | OH | Angelovl | OH |

Comfrey is an herb that contains allantoin which is a cell proliferant that helps speed up the natural replacement of body cells so as to promote the healing of damaged or injured tissues, as well as maintaining cell growth and helps prevent diseases. The allantoin in Comfrey helps stimulates reproduction of cells and promotes the formation of new tissue. Comfrey can be useful to help treat a wide variety of ailments ranging from bronchial problems, broken bones, sprains, arthritis, gastric and varicose ulcers, burns, acne and other skin conditions. Comfrey can also be useful to promote healthier bones and teeth.

Comfrey is a demulcent, mildly astringent and expectorant plant. Comfrey is frequently used as a mucilaginous medicine and has been used like marshmallow for intestinal troubles. Comfrey can be used to help treat diarrhea and dysentery. The demulcent action of Comfrey has also been used to help treat lung (pulmonary) troubles and whooping-cough. Comfrey can be further used as a strong decoction or tea to help treat internal hemorrhage from the lungs, stomach, bowels or from bleeding piles.

Comfrey leaves can be used as a topical or external remedy to help treat sprains, swellings, bruises, severe cuts, boils, abscesses, and ulcers. Comfrey leaves can also be helpful to reduce inflammation, treat gout, and heal superficial blood clots in the legs (thrombophlebitis). Internally, the Comfrey leaves can be taken in the form of an infusion. The Comfrey root is the part of Comfrey that is typically used to help treat coughs. The entire Comfrey plant can be useful for soothing pain in an inflammatory swelling.

Comfrey can help promote the healing of bruised and broken bones and can hasten the callus of bones under repair. Comfrey can be helpful to treat external ulceration as well as ulcers of the stomach and duodenum. Comfrey can further be useful to help treat ruptures, hemorrhoids, and gangrenes, and ease (relieve) pained joints.

The root portion of Comfrey can also be used as a mouthwash and gargle for sore throat and gum disease. Taken internally, the Comfrey root has also been used as a remedy for stomach aches, rheumatism, chest congestion, diarrhea, and inflammation in the lining of the lungs.

Comfrey can be useful to topically treat wounds and to reduce the inflammation associated with sprains and broken bones. The roots and leaves of Comfrey contain allantoin which as previously described is a substance that promotes wound healing and tissue regeneration. Advantageously, Comfrey ointments and preparations can be applied to the surface of the skin to heal bruises as well as pulled or injured muscles and ligaments, fractures, sprains, and strains. Comfrey can be particularly effective in slow healing wounds and to help repair tissue damage. Comfrey can also be useful as a topical preparation for psoriasis, eczema, sores, varicose veins and ulcers, arthritis, sprains, bunions, hemorrhoids, sore breasts during lactation. Comfrey contains an excellent cell proliferant and can be used to stimulate growth of new skin cells.

### Elder

Elder has a botanical name of Sambucus nigra (LINN) and is from the family of N.O. Elder is also known as Sambucus, Bourlrce flowers. Black or European cider (England), Holunderbliiten, Aalhornbliiten, Fliedertee. Schwitztec (Germany), Fleurs de sureau (France), Black Elder., Common Elder, Pipe Tree. Bore Tree, Bour Tree, Hylder, Hylantree. (Anglo-Saxon) Eldrum. (Low Saxon). Ellhorn. (German) Hollunder. (French) Sureau. Elder is obtained from the plant Sambucus nigra L., cider (Caprifoliaccac). Elder is grown throughout Europe, West and Central Asia, and North Africa, and can be obtained from the former USSR, former Yugoslavia, Bulgaria, Hungary, Romania, and the UK.

Elder is a bitter, pungent, cooling herb. Both the flowers and leaves contain rutin, isoquercitrin and hyperoside, while the flowers also contain chlorogenic acid, organic acids and triterpenoids, such as a-amyrin and b-amyrin, and the fruits contain tannins and anthocyanins such as sambucin, sambucyanin, chrysanthemin and all the glycosides of cyanidin. The seeds on the other hand contain cyanogenic glycosides such as sambunigrin, prunasin and holocalin, while the fruit contains lectins.

In particular, the composition of Elder comprises: 0.03%-0.14% essential oil of a buttery consistency, comprising a high proportion of free fatty acids (66%, main component palmitic acid) and C₁₄₋₃₁ 11-alkanes (7.2%); so far, 63 components have been identified. Ca. 1.8% flavonoids, almost exclusively flavonols and their glycosides, with rutin as the chief component (up to 1.92%), and also isoquercitrin, hyperoside, astragalin, and quercitrin; 3% chlarogenic acid: p-coumaric acid, caffeic and ferulic acids and their fi-glucose esters; traces of mandelonitrile, 6-glucoside (sambunigrin); triterpenes: fatty-acid esters; triterpene acids: 0.85% ursolic and oleanolic acids, fi-hydroxyursolic acid; 0.11% sterols, free, esterified, and glycosidic; mucilage, and tannins.

The bark of Elder is a strong purgative which can be used in an infusion as an emetic, purgative, a diuretic, and for cleaning blood. An aqueous solution of Elder has been found very useful in cardiac and renal dropsies, as well as to help treat epilepsy. An important constituent of Elder flowers is a trace of semisolid volatile oil, present to the extent only of 0.32% by weight, possessing the odor of the flowers. Elder flower in water can be used for treatment of the eye and for skin lotions. Elder flower in water is mildly astringent and a gentle stimulant. Elder vinegar is made from the flowers and can be used to help treat a sore throat. Elder berries can have aperients, diuretic and emetic properties, and the inspissated juice of the berries can be used to treat rheumatism and syphilis and as a laxative. Elder tea can be helpful to treat colic and diarrhea. Elder wine can also be helpful to treat shivering and sore throat.

Elder flowers can have diuretic, laxative, anti-inflammatory, and antiviral properties. Elder flowers can be useful in an ointment or medicinal preparation for dressing wounds, burns and scalds, as irritated and inflamed skin and minor injuries. Elder flowers can also be useful to treat influenza, mouth ulcers colds, mucus, sinusitis, feverish illnesses and other upper respiratory tract problems, as well as hay fever.

Elder fruits can have antioxidant and diuretic properties. Elder fruits can be useful for their analgesic effects, as well as their diuretic, laxative and diaphoretic properties.

Elder bark can be useful to help treat constipation and arthritic conditions.

Elder leaves can be used in an ointment or a medicinal preparation to help treat bruises, sprains, and wounds. Elder leaves can also be useful in insecticides.

The flowers and fruits of Elder can be used to lower fever, reduce inflammation, and sooth irritation.

### Hops

Hops have a botanical name of Humulus Lupulus (LINN) and are from the family N.O. Urticaceae. Hops are sometimes known as Humulus. Lupulus (English.), Hopfen. Hopfenzapfen, Hopfendriisen (German). Cones d'houblon, and Lupulin (French). Hops are grown in United States, Great Britain, Germany, France, Russia, Australia, New Zealand, Europe, India, and China. Hops can be cultivated with deep, rich soil, on dry bottom, and free circulation of air.

The root of Hops is stout and perennial, long, flexible, tough, angled and prickly, with a tenacious fiber. The leaves of Hops are heart-shaped and lobed, on foot-stalks, and are usually placed opposite one another on the stem, though sometimes the upper leaves are arranged singly on the stem, springing from alternate sides. Hops leaves are a dark-green color with their edges finely toothed. The flowers of Hops spring from the axils of the leaves. Hops are dioecious, i.e. male and female flowers are on separate plants. The male flowers of Hops are in loose bunches or panicles, 3 to 5 inches long. The female flowers of Hops are in leafy cone-like catkins, called strobiles. When fully developed, the strobiles are about 1¼ inch long, oblong in shape and rounded, consisting of a number of overlapping, yellowish-green bracts, attached to a separate axis. If these leafy organs are removed, the axis will be seen to be hairy and have a zigzag configuration. Each of the bracts of Hops enfolds at the base a small fruit (achene) The fruit and bract of Hops are sprinkled with yellow translucent glands, which appear as a granular substance. Much of the value of Hops depends on the abundance of this powdery substance, which contains 10% of Lupulin, the bitter principle to which Hops owe much of their tonic properties.

The parts of Hops that can be used as an herb or drug include: (a) The strobiles, collected and dried; and (b) The Lupulin, separated from the strobiles by sifting.

Hops have an aromatic odor due to a volatile oil (0.3% to 1.0%) comprising sesquiterpene Humulene. Petroleum spirit extracts (7% to 14%) comprising an antiseptic soft resin, and ether extracts a hard resin. The petroleum spirit extract contains the two crystalline bitter principles (a) Lupamaric acid (Humulone) and (b) Lupamaric acid (Lupulinic acid). These materials are primarily contained in the glands at the base of the bracts. The leafy organs contain about 5% of tannin. The bitter substances (acylphloroglucides) present in the resin (15-30% in Hops, 50-80% in hop rains) which are differentiated into the light-petroleum-insoluble part (hard resin) and the light-petroleum soluble (soft resins). An important component of the a-soft resin is the bitter substance humulone. The soft resin contains mainly lupulone, another bitter substance. Many other bitter substances have been isolated in pure form. All the bitter substances are usually slowly converted to components of the hard resin (mainly oxidation products). Hops can also comprise: essential oil (in Hops 0.3-1%, in hop grains 13%) mainly, mono- and sesquiterpenes (myrcene, linalool, farnesene, caryophyllene, tannins (1-4% in Hops, little in Hops grains) and flavonoids (kaempfer-01 and quercetin mono- and diglycosides). including xanthohumol and other ehaleones, as well as small amounts of phenol-carboxylic acids (ferulic and chlorogenic acids. etc.) can be present.

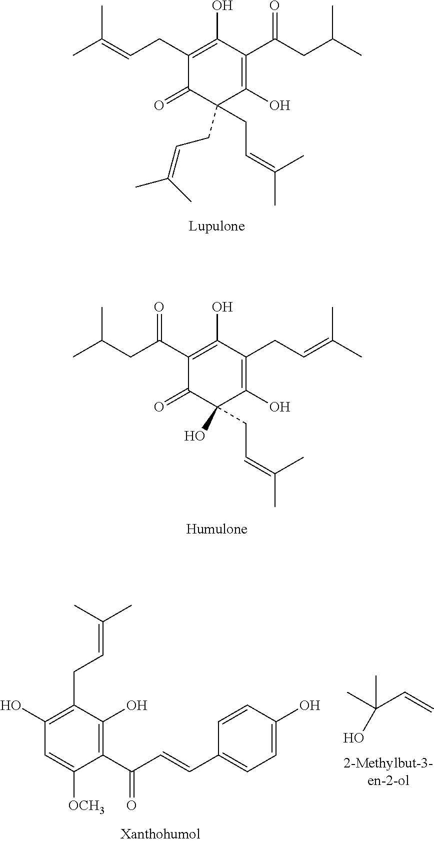

Fresh Hops possess a bitter aromatic taste and a strong characteristic odor ascribed to oxidation of the soft resin with production of Valerianic acid. Lupulin, which comprises glandular powder present on the seeds and surface of the scales, can be separated by shaking the strobiles. The Hops drug occurs in a granular, brownish-yellow powder, with the strong odor and bitter aromatic taste characteristic of Hops. The glands readily burst on the application of slight pressure and discharge their granular oleo-resinous contents. The primary constituent of Lupulin is about 3% of volatile oil, which primarily comprises Humulene, together with various oxygenated components to which the oil owes its peculiar odor. Other constituents comprise Lupamaric acids, cholene and resin.

Hops can have tonic, nervine, diuretic and anodyne properties. The volatile oil of Hops can produce sedative and soporific effects, and the Lupamaric acid or bitter principle in Hops can be stomachic and tonic. Hops can improve the appetite and promote sleep. The preparations of Hops can be by an infusion or a tincture. The infusion can be used as a vehicle, especially for bitters and tonics. The tincture is stomachic and can be used to improve the appetite and digestion. Both preparations have been considered to be sedative. Hops can also help alleviate nervousness and hysteria and induce sleep. Hops can also help treat heart disease, fits, neuralgia and nervous disorders, and can be a useful tonic to help treat indigestion, jaundice, and stomach and liver ailments, as well as irritable bladders. Hops can often relieve toothaches and earaches and alleviate nervous irritation. Lupulin in Hops is an aromatic bitter and is reputed to be mildly sedative, inducing sleep without causing headache.

In particular, Hops can be used as a sedative, especially in the form of the extract, and can be combined with other sedative drugs, to treat restlessness, hyper excitability, nervous insomnia, and stress states. Hops in the form of infusions are also used as a bitter and stomachic to stimulate the appetite and to increase gastric secretion. Hops can be used internally for nervous tension, insomnia, anxiety, irritability, nervous digestion (including irritable bowel syndrome) and premature ejaculation. Hops can help promote sleep and decrease the desire for alcohol.

Significantly, Hops can be used as an infusion to treat sores and skin injuries and internally for inflammation of the bladder. Hops can be used externally and topically for skin infections, eczema, herpes and leg ulcers. The antiseptic and seborrhoeic properties of Hops can be used in shampoos for greasy hair and in dandruff treatments. Hops can also be incorporated into bath gels because of the action as stimulants of the cutaneous metabolism and it has recently been included in the group of products which enhance hair growth.

### Echinacea

Echinacea is a genus of nine species of herbaceous plants in the Family Asteraceae. All are native to eastern and central North America. In is also cultivated in the United Kingdom. The genus name is from the Greek echino, meaning “spiny”, due to the spiny central disk. Echinacea species are perennials which belong to the Aster family. There are 9 different types (species) of Echinacea of the family Astericaea, namely, Echinacea angustifolia (Echinacea augustofolium)—Narrow-leaf Coneflower; Echinacea atrorubens (Echinacea atribactilus)—Topeka; Purple Coneflower; Echinacea laevigata (Echinacea vegetalis)—Smooth Coneflower or Smooth Purple Coneflower; Echinacea pallida (Echinacea pallidae) (Echinacea pallidum),—Pale Purple Coneflower; Echinacea paradoxa—Yellow Coneflower or Bush's Purple Coneflower; Echinacea purpurea—Purple Coneflower or Eastern Purple Coneflower; Echinacea sanguinea—Sanguine purple coneflower; Echinacea simulata—Wavyleaf Purple Coneflower; Echinacea tennesseensis—Tennessee Coneflower; and cultivars. Of nine Echinacea species, only three are used for medicinal purposes (Echinacea angustofolia, Echinacea pallida, and Echinacea purpurea).

Echinacea is also known as an American coneflower, black Susan, black Sampson, combflower, hedgehog, Indian head, Kansas snakeroot, purple coneflower, scurvy root, snakeroot, Niggerhead. Rudbeckia. Brauneria pallida (Nutt.), cock-up-hat, combflower, hedgehog, igelkopf, Indian head, kegelblume, red sunflower, rudbeckia, solhat, and sun hat.

Echinacea plants are herbaceous and drought-tolerant perennials growing to 1 or 2 m in height. Echinacea plants have tall stems, bear single pink or purple flowers and have a central cone that is usually purplish-brown in color. The large cone is actually a seed head with sharp spines that resemble a stiff comb. Echinacea leaves are lanceolate to elliptic, 10-20 cm long and 1.5-10 cm broad. Echinacea bark is thin. Echinacea wood, is thick, in alternate porous, yellowish and black transverse wedges, and the rhizome has circular pith.

Echinacea flowers bloom from early to late summer. Echinacea flowers are a composite inflorescence, with purple (rarely yellow or white) florets arranged in a prominent, somewhat cone-shaped head with the petals of the outer ray florets tend to point downward once the Echinacea flower head opens, thereby forming a cone. Echinacea flowers have a faint aromatic smell, with a sweetish taste, leaving a tingling sensation in the mouth.

The aboveground parts of the plant and roots of Echinacea can be used fresh or dried to make teas, squeezed juice, extracts, or preparations for external use.

The phytochemicals in the botanical Echinacea have demonstrated impressive activity against bacteria, viruses, and some fungi. Echinacea contains active substances that can enhance the activity of the immune system, reduce inflammation, and have hormonal, antiviral, and antioxidant effects. Echinacea can assist in the production of interferon and increases antiviral activity. Echinacea may reduce the severity of symptoms and reduce the duration of illness. Echinacea can improve the migration of white blood cells to attack foreign microorganisms and toxins in the bloodstream. Echinacea can also be an effective therapeutic agent in many infectious conditions.

Echinacea can be used externally or topically, preferably with other phytochemicals or compounds, to help treat various ailments, maladies, symptoms, and conditions, such as: (1) herpes, (2) acne and blemishes, (3) psoriasis, (4) gargles for sore throats, (5) wounds, (6) burns, (7) ulcers, (8) skin inflammations, (9) improve skin tone, (10) dry and cracked skin, (11) upper respiratory problems, (12) boils, (13) psoriasis, (14) eczema, (15) inflammatory skin conditions, (16) relieve pain, (17) insect bites, (18) arthritis (19) lymphatic swelling, (20) staph infections, (21) strep infections, (22) ear infections (also known as otitis media), (23) athlete's foot, sinusitis, and (24) hay fever (also called allergic rhinitis).

Echinacea can be used internally, preferably with other phytochemicals or compounds, to help treat various diseases, ailments, maladies, symptoms, and conditions, such as: (1) coughs, (2) common colds, (3) influenza (flu), (4) stimulate the immune system, (5) bronchitis and respiratory infections, (6) urinary infections, (7) chronic infections, (8) skin diseases, (9) fungal infections, (10) septicemia, (11) slow-healing wounds, (12) chronic fatigue syndrome, (13) venereal diseases, (14) enlarged lymph glands, (15) HIV, (16) hemorrhoids, (17) and a diphtheria, (18) putrid fevers, (19) erysipelas, (20) septicemia, (21) syphilis, (22) vaginal yeast (candida) infections (23) gastroenteritis, (24) weight loss, and (25) stimulate the production of white blood cells. Echinacea can increase bodily resistance to infection

Herbalists consider Echinacea one of the best blood purifiers and an effective antibiotic. Echinacea can activate the body's immune system increasing the chances of fighting off any disease.

People are more likely to experience allergic reactions to Echinacea if they are allergic to plants in the daisy family, which includes ragweed, chrysanthemums, marigolds, and daisies. Also, people with asthma or atopy (a genetic tendency toward allergic reactions) may be more likely to have an allergic reaction when taking Echinacea.

The Plains Indians used various species of Echinacea to treat poisonous insect and snake bites, toothaches, sore throat, wounds, as well as mumps, smallpox, and measles. Echinacea angustofolia rhizome was used by North American Plains Indians, perhaps more than most other plants, for various herbal remedies. Echinacea was one of the basic antimicrobial herbs of Eclectic medicine in the mid 1800s through the early 1900s and was used for snakebite and anthrax. Echinacea has become popular in both Europe and America as an herbal medicine.

Echinacea purpurea is a species of an herbaceous plant of the genera Echinacea, which is one of three genera of the family Asteraceae. Echinacea purpurea has a long reported use for its therapeutic and medicinal effects and is commercially available in dry powder form. Some of these reported uses include topical treatment of snake bites, infected wounds, and intra-oral treatment of a sore throat.

The phytochemicals in the botanical Echinacea, and particularly Echinacea purpurea, have demonstrated impressive activity for use in help treating various symptoms, maladies, ailments and diseases. Echinacea purpurea is the preferred type of Echinacea for this application.

The constituents of Echinacea include essential oil, polysaccharides, polyacetylenes, betaine, glycoside, sesquiterpenes and caryophylene. Echinacea also contains copper, iron, tannins, protein, fatty acids and vitamins A, C, and E. The most important immune-stimulating components are the large polysaccharides, such as inulin, that increase the production of T-cells and increase other natural killer cell activity. Echinacea also contains fat-soluble alkylamides and a caffeic acid glycoside called echinacoside that further help contribute to the herb's immune empowering effects.

A phytochemical concentrate composition of Echinacea purpurea comprises the following isolated constituents, botanical extracts, microbial inhibitors, and antimicrobial isolates: polysaccharides, echinacen, echinaceine, echinacoside (caffeic acid ester), echinolone, echinadiol, enzymes, glucuronic acid, inuloid, pentadecadiene, polyacetylene compounds, arabinogalactan, rhamnose, PS I (a 4-0-methylglucoronoarabinoxylan, Mᵣ 35 kD) and PS II (an acid rhamnoarabinogalactan, Mᵣ 450 kD), cynarin (1,5-di-0-caffeoylquinic acid), chicoric acid (2,3-0-di-caffeoyltartaric acid) and derivatives, alkylamides, keto-alkynes and -alkenes; quinones; oils including: borneol, bornyl acetate; pentadeca-8(z)-en-2one, germacrene D; caryophyllene; caryophyllene epoxide; anthocyanins pyrrolizidine alkaloids; lipophilic amides, isobutylamides; polyacetylenes; myrrha gum resin; curzerenone (furahoeudesmane type); dihydro fuanodien-6-one; 2-methoxyfuranodiene (furanoelemene type); elamol; lyndestyrene (furanogermacrane type); alkylamides, apigenin, arabinogalacta, ascorbic acid, behenic-acid-ethyl-acid, betaine, borneol, bornyl-acetate, caffeic-acid, 2-0-caffeoyl-3-(5-alpha carboxybeta) 3,4 dihydroxyphenyl, 2-0-caffeoyl-3-0 cumaroyltaraic acid, 6-0-caffeoylechinacoside, 2-0-caffeoyl-3-0-feruloyltartaric acid, 2-0-caffeoyltartaric acid, calcium, carbonate, beta carotene, carophyllene, carophyllene-epoxide, chloride, chlorgenic acid, cichoric acid, cichoric-acid-methyl-ester, cobalt, cyanadin-3-0-(beta-d-glycopyranoside), cynadin-3-(6-0-malonyl beta-d-glycopyranoside), cynarin, deca (2e,4e,6e) trienoic acid-isobutylamide, des-rhamnosylverbascoside, 3,5-dicaffeoylquinic acid, 4-5-0 dicaffeoylquinic acid, 2,3-O-diferuloltartaric acid, do-deca-(2e,4e)-dienoic acid-isobutylamide, dodeca-2,4-dien-1-yl isovalerate, dodeca (2e,6z,8e,10e)-tetraenoic acid-isobutylamide, epishobunol, beta-farnesene, 2-0-feruloytartaric acid, germacrene, heptadeca-(8z,11z)-dien-2-one, heteroxylan, humulene 8-12,(e)-10-hydroxy-4,10-dimethyl 4,11-dodecadien-2-one, 13-hydroxyoctadeca-(9z,11e,15z)-trienoic-acid, inulin, iron, isochlorogenic acid, isorhamnetin-3-rutinoside, isotussilagine, kaempferol, kaempferol-3-glucoside, kaempferol-3-nutinoside, limonene, luteolin, luteolin-7-glucoside, magnesium, manganese, 2-methyltetradeca-5,12diene, 2-methyltetradeca-6,12dience, methyl-p-hydroxycinnamate, marcene, niacin, palmitic acid, pentadeca-(8z,11z)-dien-2-one, pentadeca-(8z,13z)-dien-11-lyn-2-one, pentadeca-8en-2-one, pentadeca-(8z)-en 2 one, pentadeca-(8z)-en-11,13dien-2-one, 1-pentadecene, penta-(1,8z)-diene, phosphorous, alpha pinene, beta pinene, polyacetylenes, pontica epoxide, potassium, protein, quercetagetin-7-glucoside, quercetin, quercetin-3-galactoside, quercetin-3-glucoside, quercetin-3-robinoside, quercetin-3-xyloside, quercetin-3-xylosylgalactoside, rhamnoarabinogalactan, riboflavin, rutin, rutoside, selenium, silicate, beta-sitosterol, sitosterol-3-beta o-glucoside, sodium, stigmasterol, sulfate, tartaric acid, tetradeca-(8z)-en-11,13dien-2-one, thiamin, n-triacontanol, trideca-1-en-3,5,7,9,10-pentayne, tussilagine, vanallin, verbascoside, sequiterpenes; acetic acid, alpha-amyrone, arabinose, alpha-bisabolene, gamma-bisabolene, cadinene, campesterol, cholesterol, cinnamaldehyde, commiferin, alpha-commiphoric acid, beta-commiphoric acid, gama-commiphoric acid, commiphorinic acid, m-cresol, cumic alcohol, cuminaldehyde, dipentene, elemol, 3-epi-alpha-amyrin, eugenol, furanodiene, furanodienone, galactose, gum, heerabolene, alpha-heerabomyrrhol, beta-heerabomyrrhol, heeraboresene, limonene, 4-0-methyl-glucuronic acid, n-nonacesane, beta-sitosterol, xylose, caropylenes (carophylenes), myrrha gum resin, curzenone, dihydro fuanodien-6-one, and 2-methoxyfurandiene.

Echinacea purpurea is the preferred type of Echinacea for this application. Echinacea purpurea can comprise by weight based upon the total weight of the composition:
from about 0.3% to about 9% by weight echinacoside;
from about 0.1% to about 7% by weight PS I (4-O-methyglucoronoarabinoxylan, Mᵣ 35 kD) and PS II (acid rhamnoarabinogalactan, Mᵣ 450 kD);
from about 0.1% to about 10% by weight cynarin (1,5-di-O-caffeoylquinic acid) and chicoric acid (2,3-O-di-caffeoyltartaric acid);
from about 0.2% to about 4% by weight echinolone;
from about 0.2% to about 8% by weight echinacin B;
from about 0.1% to about 6% by weight echinaceine;
from about 2% to about 7% by weight anthonocyanins comprising cyanidin 3-O-B-D-glucopyranoside and 3-O-(6-O-malonyl-B-D-glucopyranoside);
from about 0.01% to about 0.06% by weight pyrrolizidine alkaloids comprising tussilagine and isotussilagine;
from about 0.003% to about 0.009% by weight isomeric dodeca isobutylamides and tetroenoic acid; and
from about 0.01% to about 2% by weight carophylenes.

The chemical formula of some of the botanical extracts of Echinacea is shown hereinafter.

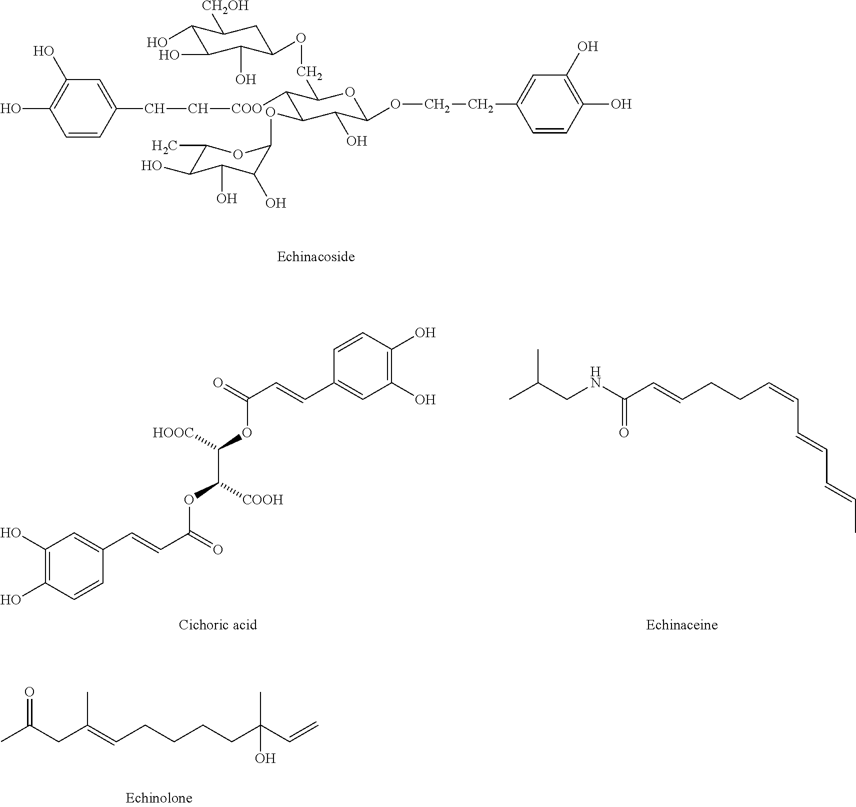

### Camphor

Camphor is from the botanical family Lduraceae and includes the genus and species Cinnamomum camphora syn. Laurus camphora. Camphor is also known Gum Camphor, Laurel Camphor, Cemphire, Alcanfor, Plumajillo, Camphor zhang nao, Cinnamomum, camphora, and Laurus camphora, 2-bornanone, 2-camphanone, and bornan-2-one. Formosa Camphor also occurs in some other related trees in the laurel family, notably Ocotea usambarensis.

The word Camphor derives from the French word camphre, as well as from Medieval Latin camfora, Arabic kafur, and Malay kapur Barus meaning “Barus chalk”. Malay traders from whom Indian and Middle East merchants would buy camphor called it kapur, “chalk” because of its white color. Barus was the port on the western coast of the Indonesian island of Sumatra where foreign traders would buy Camphor. In the Indian language Sanskrit, the word ‘karpoor’ is used to denote Camphor. A south Indian adaptation of this word, ‘karpooram’ has been used for Camphor in many south-Indian or Dravidian languages, such as Telugu, Tamil, Kannada and Malayalam.

Camphor is a terpenoid with the chemical formula of C₁₀H₁₆O and a chemical name of 1,7,7-trimethyl-bicyclo[2,2,1]heptan-2-one; 2-Camphanone or 1,7,7-Trimethylbicyclo[2.2.1]-2-heptanone. Camphor has a melting point of 179.75° C. (452.9 K) and a boiling point of 204° C. (477 K). Camphor is a waxy, white or transparent crystalline solid with a strong, aromatic odor. Camphor is found in wood of the camphor laurel and Cinnamonum camphora. Camphor can also be synthetically produced from oil of turpentine.

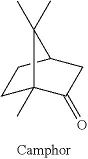

Camphor is native to China and Japan. Camphor trees now thrive in Egypt, Taiwan (Formosa), Borneo, Madagascar, India, Ceylon, Canary Islands, Argentina, Brazil, southern Europe, and the United States. Camphor is cultivated in tropical and subtropical regions for its wood, from which the medicinal oil is derived. Natural commercial Camphor typically comes from C. camphora and Dryobalanops camphora (fam. Dipterocarpacaea) (Borneo Camphor).

Camphor trees are evergreen and have pointed shiny leaves give off a distinctive Camphor aroma. Camphor evergreen trees can reach 100 feet. Camphor trees produces red leaves that turn yellow or dark green. Camphor leaves can be about 15 cm long. Camphor trees bear flowers in summer that appear in clusters and are small and yellow to white. Camphor flowers are followed by small (up to 9 mm wide) black fruits or red berries. Camphor trees generally grow slowly.

The D. aromatica tree, found in Sumatra and Borneo, grows to an enormous height, often over 100 feet, and trunk 6 or 7 feet in diameter. The Camphor of the older trees exists in masses, in longitudinal cavities, in the heart of the tree, 1½ feet long at certain distances apart. The usual way of finding out if Camphor has formed in the tree is by incision. This Camphor is chiefly used for funeral rites, and any that is exported is bought by the Chinese at a high price, as they use it for embalming, it being less volatile than ordinary Camphor.

Another Camphor called N'gai, obtained from the Blumea Balcamferi (Compositae), differs chemically from the Borneo species, levogyrate, and is converted by boiling nitric acid, to a substance considered identical with stearoptene of Chrysanthemum parthenium. The N'gai, plant grows in Great Britain and is also known as a Double-flowered Bush Fever-Few.

Many Chinese believe that Camphor cannot be extracted from trees under 50 years old. In the United States, Camphor is extracted from leaves and twigs of the oldest trees, which does less damage than the more invasive Chinese method. When the Camphor root or bark is steamed, it produces a volatile, white, crystalline compound with a characteristic pungent odor, usually referred to as Camphor.

Much of the Camphor, however, now used in the United States is produced synthetically. Camphor was first synthesized by Gustaf Komppa in 1903. The first industrial total synthesis of Camphor occurred in 1907 when Gustaf Komppa began industrial production in Finland.

Historically, in the 13th century, Marco Polo noted that Camphor oil was highly valued by the Chinese as a medicine, scent, and embalming fluid. The Spanish name for the Camphor plant comes from the Arab “alkafur,” which suggests that Camphor was part of Spanish or Moorish medicine. Camphor has also been used for many centuries as a component of incense.

Two substances are found in commerce under the name of oil of Camphor: one is the product of C. cinnamonum, and is known as Formosa or Japanese oil of Camphor; the other as East Indian oil of Camphor, from the D. aromatica. East Indian Camphor oil is less volatile than Japanese Camphoroil, and has a distinctive odor; East Indian Camphor oil is highly prized by the Chinese, who use it for embalming purposes and to scent soap. The Chinese attribute many virtues to East Indian Camphor oil. Recently, large quantities of Camphor oil have come into the American and European markets as Japanese oil; it varies in quality and color from a thin watery oil to a thick black one. Camphor can be imported in tin cans and varies greatly in the amount of Camphor it contains; some cans having had all the solid principle extracted before importation. The odor of Camphor is peculiar, like sassafras and distinctly camphoraceous. Camphor oil is used in Japan for burning, making varnish and for Chinese inks, as a diluent for artists' colors; it has a better capability for dissolving resins that Turpentine.

Medicinally, Camphor can be used in topical ointments, liniments, and creams and can be useful to relieve mild pain and itching. Camphor can be useful to help relieve irritation of the sexual organs and for gout. Camphor is an active ingredient in the well-known commercial product sold under the brand name Vicks VapoRub. Camphor can readily be absorbed through the skin and produces a feeling of cooling. Camphor can function as a local anesthetic to numb the peripheral sensory nerves, and can be slightly antiseptic. Camphor can also be effective as a cough suppressant. Topically, Camphor can be used to relieve pain from cold sores, hemorrhoids, warts, and minor burns. Camphor can be useful for backache medications and to help treat chapped lips and skin conditions such as acne and oily skin. Camphor can further be uses externally as a counter-irritant in rheumatisms, bronchitis, and in inflammatory conditions. Camphor can further be used in acne medications, ear drops, antimicrobial substance, to treat bruises and sprains, and to help soothe the skin. The U.S. Food and Drug Administration (FDA) has recognized Camphor as a safe and effective topical antitussive, analgesic, anesthetic, and antipruritic agent. Camphor can have antiseptic, anti-irritant, carminative, circulatory, as well as analeptic properties.

Camphor can be used internally or orally because of its calming influence in hysteria, nervousness and neuralgia, and for diarrhea. Camphor can also be used as a stimulant for blood circulation and the nervous system, as well as for minor heart symptoms and fatigue. Camphor can also help treat colds, chills, coughs, bronchitis, fever, insomnia, rheumatism, fever and for various inflammatory complaints. Camphor can further enhance digestion and to kill internal parasites.

Camphor oil can be useful as a stimulant and to help alleviate pains and adverse conditions in the stomach, bowels, spasmodic cholera, and flatulent colic. Camphor oil can also be used as a sedative liniment and if diluted with olive oil or soap, Camphor oil can be useful to help treat rheumatism, sprains, bruises, and joint. Camphor has been approved by the German Commission for external use in the treatment of muscular rheumatism and inflammatory diseases of the respiratory tract. Camphor oil has analgesic, anti-depressant, anti-spasmodic, cardiac, carminative, diuretic, hypertensive, insecticide, laxative, and stimulant properties.

Camphor can be used in the manufacture of many other products, including celluloid, cellulose nitrate, explosives, fireworks, insecticides, moth repellents, and soaps. 1 Camphor can also be used for its scent, as an embalming fluid. Camphor may also deter snakes and other reptiles due to its strong odor.

Historically, Camphor has been used by the Chinese for embalming, as an ingredient in soap, for wounds and skin diseases, and as a stimulant in unconsciousness. In the 17th Century, Camphor may have been used by Auenbrugger for treatment of manial.

### Hypericum Perforatum

### St. John's Wart

Hypericum Perforatum (St. John's Wart) (St. John's Wort), is also referred to as “Hyperici herba”, as well as Hypericum, Johanniskraut, Blutkraut, Herrgottsblut, Walpurgiskraut, l-lexellkraut (German), Herbe de millepertuis (France). St. John's Wart can be obtained from the plant Hypericum perj., common or perforatex. St. John's wart is located in eastern Europe and western Asia.

From a chemical viewpoint, St. John's Wart comprises 0.3% hypericin and hypericin-like substances, notably pseudohypericin, isohypericin, protohypericin. etc. The northern European broad-leaved var. perforatum tends to have less of the hypericins than the southern Europe. St. Johns Wart also comprises flavonoids: especially hyperoside (“hyperin”) and rutin, as well as biflavones, particularly biapigenin. Among the antibiotic substances, St. John's Wart has up to 3% hyperforin oil (n-alkanes, especially C₂₉H₆), also Ct.-pinene and other monoterpenes). St. Johns Wart further comprises 0.3% essential and at least 10% tannins, as well as small amounts of procyanidins.

St. John's Wart is a bittersweet herb that has cooling and astringent properties, and can be useful to calm the nerves, reduce inflammation and promote healing. St. John's Wart can be beneficial to treat psychogenic disturbances, insomnia, anxiety, or depression, e.g. during menopause and in nervous exhaustion. It is believed that hypericins as well as biflavones and hyperforin are involved in the sedative effect of St. John's Wart. St. John's Wart can also be useful as a monoamineoxidase inhibitor.

St. John's Wart can be used externally as a topical preparation because of its anti-septic and analgesic effect to help treat burns, bruises, sores, wounds, sprains, tennis elbow and cramps. Advantageously, St. John's Wart can also be useful for pain relief and inflammation caused by nerve damage. St. John's Wart can further be used to help treat shingles, sciatica and fibrositis.

St. John's Wart can be helpful when taken internally or orally, preferably with other chemical compounds. St. John's Wart can be useful to prevent or relieve diarrhea (tannin content) or diuretic (llavonoid content). St. John's Wart can also be useful for decreasing bedwetting, rheumatism, and gout. Furthermore, St. John's Wart can be useful to treat inflammation of the stomach and intestines as well as internal worms.

### Aloe Vera

Aloe is from the botanical family Aloeceae and includes the genus and species Aloe Vera and Aloe Barbadensis. Aloe Vera is also known as Aloes (England and France), Curacaodoe, and Venezuela-Aloe (German.), as well as Barbados Aloe, Cape Aloe, Curaiao Aloe, Socotrine Aloe, and Zanzibar Aloe.

Barbados Aloe comprises juice from the secretor cells of the leaves of the aloe plant which has been concentrated and allowed to solidify. Barbados Aloe is a dark brown, slightly shiny, opaque mass with a waxy conchoidal fracture. The powder is brown and soluble in warm ethanol, partly soluble in boiling water, and practically insoluble in ether and chloroform. The aqueous solution turns red after the addition of alkalis (Rorolrliger reaction).

Aloe, native to Africa, is also known as “lily of the desert”, the “plant of immortality”, and the “medicine plant”. The name was derived from the Arabic alloeh meaning “bitter” because of the bitter liquid found in the leaves. There are over 500 species of Aloe growing in climates worldwide. Ancient Greeks, Arabs and Spaniards have used the plant throughout the millennia. Africans sometimes rub the Aloe gel on their bodies to reduce perspiration and their scent.

Aloe Barbados (Barbados Aloe) is native in Africa and is cultivated particularly in the West Indies and in the coastal regions of Venezuela. Barbados Aloe is mostly exported via Curacao. Aloe Imrbadensis has been cultivated in Florida, Texas, mainly for the exploitation of the Aloe Vera gel used in cosmetics and beverages.

Aloe Vera is a clump forming perennial with rosettes of thick, fleshy, spiky, gray-green leaves, young specimens being red spotted and the plant bears yellow tubular flowers in summer. The inside of the Aloe leaves are used (also referred to as gel) as well as Aloe juice extracted from the pulpy inside and a yellow bitter laxative principle (Aloe vera latex) which is an exudate from the leaf which can dried to a crystalline substance. In particular, Aloe Vera gel is the naturally occurring and undiluted gel obtained by stripping away the outer layer of the Aloe leaf. Aloe Vera concentrate or extract is the product obtained when removing the water from the gel. Aloe Vera juice is a drink for internal use that can comprise at least 50% of the pulped Aloe gel. Aloe vera latex or bitter principle is a bitter yellow liquid derived from the pericyclic tubules of the rind of the Aloe and which primary constituent is aloin.

Aloe has anti-inflammatory, astringent, emollient, anti fungal, antibacterial and antiviral properties. Aloe contains many compounds that are biologically active and includes anthraquinones, saccharides and prostaglandins as well as other constituents. The anthraquinones contained in Aloe, includes aloin (barbaloin), isobarloin, anthranol, Aloetic acid, anthracene, ester of cinnamic acid, Aloe-emodin, emodin, chrysophanoic acid, ethereal oil as well as resistannol. Aloe contains various saccharides including glycoprotein, mucopolysaccharide and polysaccharides such as galactose, xylose, arabinose, acetylated mannose as well as the remarkable acemannan.

Aloe contains enzymes (oxidase, amylase, catalase, lipase and alkaline phosphatase) amino acids (lysine, threonine, valine, methionine, leucine, isoleucine and phenylalanine) vitamins (vitamin b1, B2, B6, C, E, folic acid, choline and beta carotene) minerals (calcium, sodium, manganese, magnesium, zinc, copper and chromium as well as other miscellaneous compounds such as cholesterol, triglycerides, steroids, uric acid, lignins, beta-sitosterol, gibberellin and salicylic acid.

The constituents of Aloe Barbados (Barbados Aloe are: Hydroxyanthracene derivatives: as the principal components (al) in Aloe capensis. q.v.) 25-40% aloins A and B; 3%-4% 7-hydroxyaloins A and B, and their 6′-p-coumaroyl and feruloyl esters, as well as small amounts of Aloe-emodin and chrysophanol. Aloe Barbados also comprises chromone derivatives: the main component (up to 30%) is 8-C-glucosylcbromone Aloe-resin B (Aloesin) and smaller amounts of the sugar-free compound Aloesone, as well as its derivative Aloeresin A and Aloeresin C.

Aloe Vera gel, which can comprise the stabilized viscous juice from the mucilage-containing parenchyma in the inner part of the succulent Aloe leaves, can be useful as an ingredient in cosmetic preparations and in some fitness or sports drinks. A wide variety of products, including creams, are manufactured with Aloe Vera since humectants, anti-inflammatory, and antibacterial actions are ascribed to the gel.

Aloe Capensis, also known as Cape Aloes, Kap-Aloe, Afrikanische Aloe, Aloes (France.), is indigenous in Africa and is cultivated in southern and eastern Africa. The commercial varieties may be classified according to their geographical origin: South Africa: Cape, Natal Aloes; East Africa/Arabia, Kenya, Uganda, Zanzibar, Mocha Aloes.

The constituents of Aloe Capensis are hydroxyanthracene derivatives comprising aloins A and B and aloinosides A and B, as well as small amounts of Aloe-emodin and chrysophanol. Aloe Capensis also comprises chromone derivatives comprising 25-40% of 8-glucosyl chromones aloeresins A and B, as well as smaller amounts of aloeresin C and iso-aloeresin A and aloeresin D Aloe capensis can further comprise 6-phenyl-2-pyrone derivatives comprising the glycoside of the bitter substance aloenin B, which is made up of aloenin A (formerly aloenin) a 6-phenyl-2-pyrone oglucoside and p-coumaroyl-glucose. The Aloe plant is about 96% water, but also contains active ingredients including essential oil, amino acids, minerals, vitamins, enzymes and glycoproteins.

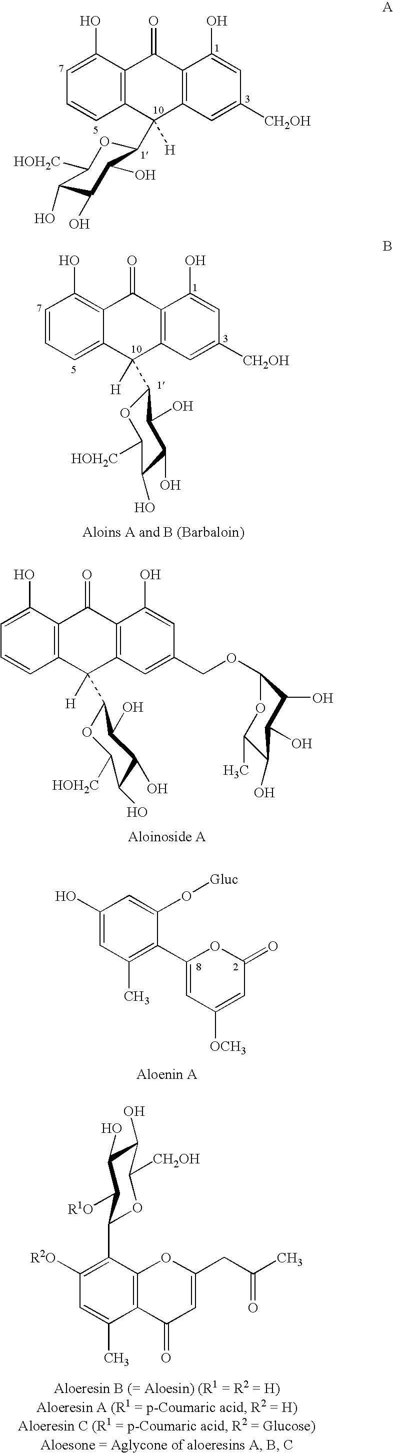

Aloe further contains at least three anti-inflammatory fatty acids that can be helpful for the stomach, small intestine and colon. Aloe can naturally alkalize digestive juices to help prevent overacidity, which is a common cause of indigestion. Aloe also helps cleanse the digestive tract by exerting a soothing, balancing effect and can be useful as a laxative

Aloe can be used internally to help treat digestive problems, such as: constipation, colitis, and irritable bowel syndrome, and to sooth the lining of the digestive tract. Furthermore, Aloe can be useful internally to help treatment asthma, diabetes, peptic ulcers caused by excess acid, aspirin, non-steroidal anti-inflammatory drugs or alcohol use as well as to stimulate and regulate various components of the immune system by stopping the inflammation and blood supply of tumors and in preventing carcinogenic compounds from entering the liver and can be combined in some cancer treatments. Aloe juice from the inner Aloe leaves can be useful for its anti-inflammatory effect on Crohn's disease. Moreover, Aloe contains aloemannan which stimulates the growth of healthy kidney cells and helps to slow the formation of kidney stones. Aloe juice can be useful to treat heartburn as well as ulcers. Aloe can be helpful in remission in skin cancers and because of its anti-oxidant effect can be effective to help prevent skin damage from x-rays and other forms of radiation. Also, Aloe-emodin may be helpful to treat herpes virus which causes cold sores and shingles.

Aloe can be used externally as a topical preparation to help treat: skin irritation, burns, scalds, sunburn, wounds, eczema, psoriasis, acne, dermatitis, and to stimulate cell regeneration. Aloe can be particularly effective for burns and other wounds because Aloe can activate the macrophages which fights bacterial infection while at the same time increasing circulation to the area which can result in accelerated healing. Aloe can help heal both traumatic wounds as well as surgical wounds because Aloe contains not only vitamin E and C as well as zinc, but also polysaccharides that help reduce inflammation and stimulate the fibroblast and epidermal growth and repair. Aloe can be an excellent, soothing treatment for the skin and can also be useful to treat hemorrhoids (piles). Aloe cream can also be useful to treat frostbite with less tissue loss and avoidance of amputation. Aloe gel can be applied directly to the skin as a softening agent. Aloe enzymes, e.g. carboxypeptidase and bradykininase, can be helpful to reduce swelling, itching, reducing inflammation as well as pain. Aloe macerated oil exhibits astringent, emollient, anti fungal, antibacterial, and antiviral properties. Aloe can also be useful with massage therapy for its healing and rejuvenating properties.

### Mistletoe

Mistletoe has a botanical name of Viscum album (LINN) and is from the family of N.O. Loranthaceae. Mistletoe is sometimes also referred to as: Folia Visci, Stipites Visci (Lat.), Viscum, European or Birdlime Mistletoe (Engl.), Mistelkraul, Vogel- or Leimmistel, Hexenbesen, Drudenfuf (German), Herbe de gui (French.), all-heal, and devil's fuge.

Mistletoe is a semi-parasite native in Europe and Asia and found on almost all deciduous trees, except beech: two subspecies occur only on conifers. Imports of the Mistletoe are available from Bulgaria, Turkey, Yugoslavia, Albania, and Russia.

Mistletoe is an evergreen parasitic plant, growing on the branches of trees, where it forms pendent bushes, 2 to 5 feet in diameter. Mistletoe is a true parasite, for at no period does it derive nourishment from the soil, or from decayed bark, like some of the fungi do—all its nourishment is obtained from its host. The root becomes woody and thick. Mistletoe is always produced by seed and cannot be cultivated in the earth like other plants, hence the ancients considered it to be an excrescence of the tree. Mistletoe will grow and has been found on almost any deciduous tree, preferring those with soft bark, and is often found on Apple trees, though it is frequently found on the Ash, Hawthorn, Lime and other trees. When one of the familiar sticky berries of the Mistletoe comes into contact with the bark of a tree—generally via birds—after a few days it sends forth a thread-like root, flattened at the extremity like the proboscis of a fly. This finally pierces the bark and roots itself firmly in the growing wood.

The stem of Mistletoe is yellowish and smooth, freely forked, and separate when dead into bone-like joints. The leaves of Mistletoe are tongue-shaped, broader towards the end, 1 to 3 inches long, very thick and leathery, of a dull yellow-green color, and arranged in pairs with very short footstalks. The flowers of Mistletoe are small and inconspicuous, are arranged in threes, in close short spikes or clusters in the forks of the branches, and are of two varieties, the male and female occurring on different plants. Neither male nor female flowers have a corolla, the parts of the fructification springing from the yellowish calyx; they open in May. The fruit of Mistletoe is a globular, smooth, white berry, ripening in December.

From a chemical viewpoint, Mistletoe comprises: Lectins (glycoproteins with the ability to bind specifically to certain sugars—. galactose or N-acetylgalaetosamine or both—and cell surfaces; 2-D electrophoresis patterns show that they each comprise a large number of isolectins which have variations in both the A and B chains, some of which have been isolated in pure form. Proteins and polypeptides, especially the viscotoxins, comprise 46 amino acids; 2-D gel electrophoresis reveals as many as 500 protein spots. Mistletoe further comprises: phenylpropanes and lignans, such as syringenin 4′-glucoside and syringaresinol 4′,4″-diglucoside; caffeicacid derivatives; flavonoids, especially derivatives of quercetin; biogenic amines (tyramine, etc.); polysaccharides, particularly galacturonans and arabinogalactans. In the leaves, the variable amount (0.9.-6.6%, based on the dry weight.) of 1 D-1-O-methyl-mucoinositol shows a marked increase in winter and may have a crypto-protective function. Mistletoe can comprise sapogenin oleanolic acid (triterpenoid saponin) and viscotoxin (cardioactive polypeptide protein).

The lectins isolated in recent years from Mistletoe, some of which are very pure, are generally composed of A and B polypeptide chains and a carbohydrate part and their principal action is a clear-cut cyototoxic effect; this has been determined for certain tumors and carcinomas (mouse ascites tumor; human tumor cells: KB and HeLa strains in tissue culture). Relevant investigations have shown that the polysaccharides probably participate in the working of the lectins. The different isolectin patterns alluded to above may indicate significant variation in the cytotoxicity and postulated immunomodulating potency of the lectins. Helixo has a cytostatic effect on in-vitro cultures of drug-resistant human acute lymphoblastic leukemia cells. Mistletoe preparations for injection, may owe their efficacy to their lectin content. Phenyl-propanes and lignans can have a significant inhibitory action towards c-AMP-phosphodiesterase.

Mistletoe contains mucilage, sugar, a fixed oil, resin, an odorous principle, some tannin and various salts. The active part of the plant is the resin, Viscin, which by fermentation becomes a yellowish, sticky, resinous mass. A homoeopathic tincture of Mistletoe can be prepared from the leaves and ripe berries.

A distinction should be made between oral use of the Mistletoe in the form of aqueous extracts, e.g. herbal teas (as an adjuvant in the treatment of high blood pressure), and the parenteral use of isolated constituents, e.g. in the form of injections. Mistletoe can be useful for treating degenerative inflammation of the joints by eliciting cuti-visceral reflexes following local inflammation brought about by injections. Mistletoe can also be useful as a palliative therapy for malignant tumors through non-specific stimulation. Mistletoe can further be used to help treat convulsions delirium, hysteria, neuralgia, nervous debility, urinary disorders, and heart disease.

### Rhus Toxicodendron

### Poison Ivy

Rhus toxicodendron (Poison Ivy) has a botanical name of Rhus Toxicodendron (LINN) in the family of N.O. Anacardiaceae. Poison Ivy is one of the species of Sumacs, an attractive group of plants widely distributed in Europe, Asia and North America, varying much in habit from low bushes to moderately-sized trees. Poison Ivy grows in thickets and low grounds in North America, where it is quite common. The root of Poison Ivy is reddish and branching; the leaves of Poison Ivy are rather large, three parted (which will readily distinguish it from the five-parted Ampelopsis). The central leaflet of Poison Ivy has a longer stalk and the lateral leaves of Poison Ivy are almost stalkless. The leaflets of Poison Ivy are entire when young, but when full-grown they are variously indented, downy beneath, thin and about 4 inches long. Poison Ivy contains an acrid juice, which darkens when exposed to air, and when applied to the skin produces inflammation and swelling. When dry, the leaves of Poison Ivy are papery and brittle, sometimes with black spots of exuded juice turned black on drying. The flowers of Poison Ivy are in loose, slender clusters or panicles and the axils of the leaves of Poison Ivy are small, some perfect, others unisexual, and are greenish or yellowish-white in color. Poison Ivy blossoms in June, and are followed by clusters of small, globular, brown colored, berry-like fruit. The sap of Poison Ivy can be extremely poisonous and in many persons the slightest contact with the leaves causes a rash. The hands and arms in some people and sometimes their whole body can become greatly swollen from touching or carrying a branch of a Poison Ivy plant. Swelling from Poison Ivy is often accompanied with intolerable pain and inflammation, ending in ulceration. Some persons however, are able to handle the plant with impunity.

Poison Ivy is sometimes referred to as Toxicodendron and was introduced into England first in 1640, but not used as a medicine until 1798, when a physician brought to his notice a young man, who had been cured of a herpetic eruption on his wrist He thereupon commenced the use of the plant in the treatment of obstinate herpetic eruptions and in palsy, many cases yielding well to the drug. Since then it has rapidly gained a place in general practice, meeting with some success in the treatment of paralysis, acute rheumatism and articular stiffness, and in various forms of chronic and obstinate eruptive diseases. Poison Ivy can be used by homoeopathists for rheumatism, ringworm and other skin disorders. Poison Ivy is also considered to be a useful remedy for treatment of Nettle rash. The fluid extract of R. Toxicodendron (Poison Ivy) can further be used as a vesicant or blister producer.

The fresh leaves of Poison Ivy can be used as an herb from which a fluid extract is prepared for use as a drug. The activity of the Poison Ivy as a drug was formerly ascribed to a fixed oil, toxicodendrol, but has been attributed more recently to a yellow resin, to which the name toxicodendrin is applied.

### Eucalyptus

Eucalyptus is also known as Eucalyptus, Blue gum, Fever tree leaves (England), Eucalyptusbliitter, Fiebcrbaumbliitter (German), Feuilles d'eucalyptus (France), or gum trees, in reference to the habit of many Eucalyptus to constantly exude sap from any break in the bark. Other names for various species of Eucalyptus include mallee, box, ironbark, stringybark, and ash. Eucalyptus is a diverse genus of trees and a few shrubs. There are more than 700 species of Eucalyptus.

Eucalyptus species are native to Australia, but come from many countries throughout the world in regions with a subtropical or Mediterranean climate. Eucalyptus dominate the tree flora of Australia, mostly native to Australia, with a much smaller number found in adjacent parts of New Guinea and Indonesia and one as far north as the Philippines. Eucalyptus can be found in almost every part of the Australian continent, adapted to all of its climatic conditions. No other continent is so characterized by a single genus of tree as Australia is by Eucalyptus.

The most readily recognizable characteristics of Eucalyptus species are the distinctive flowers and fruits. The name Eucalyptus, from the Greek words eu-, well, and kaluptos, cover, meaning “well-covered”, describes the bud cap (botanically called an operculum). This cap forms from modified petals and falls off as the flower opens. Thus flowers have no petals, decorating themselves instead with many showy stamens. The woody fruits, known as gumnuts, are roughly cone-shaped and have valves at the end which open to release the seeds.

The Eucalyptus or blue gum tree has a wide spread crown and the trunk has smooth, white bark, which peels to reveal a red shade. The leaves are silver-green when young, ranging to green when adult and differ in shape, from lance-like to sickle shaped. White flowers appear in spring and summer.

Nearly all Eucalyptus trees are evergreen but some tropical species lose their leaves at the end of the dry season. The leaves of Eucalyptus are covered with oil glands. The copious oils produced are an important feature of the genus. Eucalypts also commonly exhibit leaf dimorphism. When young, the leaves of Eucalyptus are opposite, oval to roundish, and occasionally without a petiole. Young Eucalyptus trees from one to a few years old have that are alternate, lanceolate to falcate (sickle-shaped), slender and pendulous with longer petioles. However there are numerous species of Eucalyptus such as E. melanophloia and E. setosa that retain the juvenile leaf form throughout their life history. The adult leaves of most species of Eucalyptus, as well as the juvenile leaves of some Eucalyptus, are the same on both sides, lacking the distinction between upper and lower surfaces shown by the leaves of most plants. Most species of Eucalyptus do not flower until adult foliage starts to appear; E. cinerea and E. perriniana are notable exceptions.

The composition of Eucalyptus comprises: 1.5%-3.5% essential oil, the composition of which differs little with the age of the leaves; the main component 54%-95% is cineole; when the leaves are steam-distilled to produce the oil, most of the cineole is recovered in the first hour. More specifically, Eucalyptus essential oil comprises 1,9-ceneole, a-pinene, p-cymene, limonene and other monoterpenoids. Eucalyptus also comprises small amounts of monoterpenes: as well as tannins, ellagitannins, about 2% triterpenes (ursolicacid derivatives) and flavonoids. Eucalyptus leaves contain sesquiterpenes (aromadendren and globulol), euglobals (derivatives of acylphloroglucin) and flavonoids.

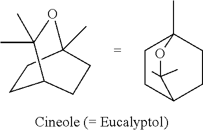

The essential oil extracted from the leaves of Eucalyptus contains compounds that are excellent natural disinfectants, but which can be toxic in large quantities. Eucalyptus also produces a lot of nectar, providing food for many insects. Several marsupial herbivores, notably koalas and some opossums, are relatively tolerant of Eucalyptus. The close correlation of these oils with other more potent toxins called formulated phloroglucinol compounds allows koalas and other marsupial species to make food choices based on the smell of the leaves. However, it is the formulated phloroglucinol compounds that are the most important factor in choice of leaves by koalas.

Eucalyptus can have analgesic, anti-rheumatic, antiseptic, anti-spasmodic, antiviral, bactericide, balsamic, cicatrizant, decongestant, deodorant, diuretic, expectorant, insecticide, rubefacient, stimulant and vulnerary properties. Eucalyptus is an aromatic herb that can have antibiotic properties. Eucalyptus can serve as a stimulant and can be used as a decongestant and expectorant that relaxes spasms and lowers fever. Eucalyptus leave extract has shown some diuretic, anti-diabetic and anti-tumor properties, and the leaves can be used for herbal tea.

Advantageously, Eucalyptus can be useful as topical preparation, liniment or ointment to help bruises, sprains and various muscular pains. Eucalyptus can also topically treat wounds and abscesses. Furthermore, Eucalyptus can help treat skin eruptions resulting from herpes, fever blisters, cuts and wounds.

Eucalyptus can be useful as an inhalation and vapor rub, to treat and relieve mucus congestion. Eucalyptus can also be useful for cough drops and decongestants. Eucalyptus can help clear the chest and treat bronchitis, sinusitis, colds and flu.

Eucalyptus oil can be used for cleaning and deodorizing. Eucalyptus can further be used in mosquito repellents and can be used as to reduce malaria by draining the soil in Algeria, Sicily and also in Europe and California. Drainage removes swamps which provide a habitat for mosquito larvae, but such drainage can also destroy ecologically productive areas.

### Commiphora Myrrha

There are different types of Commophora (also sometimes referred to as “Commiphora”), such as: Commophora myrrha, Commophora molmol, Commophora erythraea, and their cultivars. In the application, Commophora myrrha, also sometimes referred to as “myrrha” is the preferred type of Commophora.

The chemical formula of some of the botanical extracts of Commiphora myrrha are shown below.

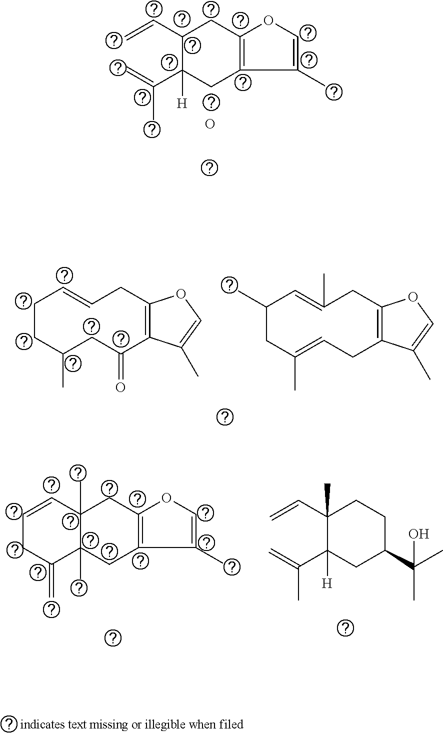

Myrrha is also sometimes referred to as: myrrh, mine, myrrhis, gummi myrrha, myrrha vera, gum myrrh, Commiphora resin, gruggal gum, gruggal resin, Heerabol myrrh, myrrhe, Manniliche myrrhe, Opopanax, and Hirabol myrrh. Myrrha can comprise gum resin obtained from cuts made in the bark of trees of the genus Commiphora myrrha, i.e. the myrrh tree. Myrrha can also comprise balsamic juices from Balsamodendron myrrha, i.e. a buraceous tree. Myrrha can also be extracted from Osmorhiza or Washingtonia, which is also sometimes referred to a sweet cicely. The myrrh tree is a native in Erythrea, Abyssinia, Somalia, Yemen, Sudan, and elsewhere.

The myrrh-producing Commiphora species are shrubs or small trees with large, sharply pointed thorns on the stem. The unequal ternate leaves are alternate and the small flowers are arranged in terminal panicles. When damaged, the schizogenous resin ducts yield the drug myrrh.

Myrrha is an air-dried oleo-gum resin that exudes from the bark of Commiphora species. The material comprises irregular, rounded grains or lumps of varying sizes with holes and range in color from dark brown and almost black to light or dark orange-brown; some parts may be yellow or colorless to pale yellow. The surface is mostly covered with a grey to yellowish grey powder; the fracture is conchoidal and yields thin, translucent fragments. Myrrha can have a sweet fragrance and a harsh and aromic order. Myrrha can have a bitter and aromatic taste. Myrrha can also be acrid and can stick to the teeth on chewing.

Commiphora molmol and other Commiphora species, insofar as the chemical composition of their gum-resin, are comparable with that of myrrha DAB 10. There is considerable confusion in the literature regarding the sources of myrrh and the identity of the Commiphora species involved. Common (or hirabol) myrrh appears to derive from Commiphora myrrha. Somalian myrrh is said to come from Commiphora molmol. However, the systematic (taxonomic) relationship between Commiphora myrrha and Commiphora molmol is not clear. The source of Abyssinian myrrh is Commiphora madagascariensis or Commiphora abyssinica. Opopanax, which is also referred to as bisabol myrrh or perfumed bdellium is believed to originate from either Commiphora erythraea (Ehrenb) or Opopanax.

The composition of myrrha is very complex and only partially known from 40-60% of myrrha is soluble in ethanol and comprises a resin and an essential oil. Myrrha contains polysaccharides, triterpenoids, triterpene acids and an essential oil containing sesquiterpenes and furano sesquiterpenes. More specifically, Myrrha consists almost entirely of sesquiterpenes. The main components of sesquiterpenes are: furanosesquiterpenes of the germacrane elemane, eudesmane, and guaiane types. In addition, there are sesquiterpene hydrocarbons, and sesquiterpene alcohols, e.g. elemol. Presumably, some of the furanosesquiterpenes are characteristic of pharmaceutical myrrh. Myrrha curde gum or crude mucilage includes 20% proteins and 65% carbohydrates which are made up of galactose, 4-0-methylglucuronic acid, and arabinose. Commophora myrrhaphyto chemicals comprise: acetic acid, alpha-amyrone, arabinose, alpha-bisabolene, gamma-bisabolene, cadinene, campesterol, cholesterol, cinnamaldehyde, commiferin, alpha-commiphoric acid, beta-commiphoric acid, gama-commiphoric acid, commiphorinic acid, m-cresol, cumic alcohol, cuminaldehyde, dipentene, elemol, 3-epi-alpha-amyrin, eugenol, furanodiene, furanodienone, galactose, gum, heerabolene, alpha-heerabomyrrhol, beta-heerabomyrrhol, heeraboresene, limonene, 4-0-methyl-glucuronic acid, n-nonacesane, beta-sitosterol, xylose, caropylenes (carophylenes), myrrha gum resin, curzenone, dihydro fuanodien-6-one, 2-methoxyfurandiene, and lynderstyrene (lindestyrene).

Macro and microscopically, myrrha can appear as a brownish yellow powder characterized by yellowish splinters or spherical grains of various sizes, along with fine granular material which swells in water. In chloral-hydrate mounts, there are only a few fragments of tissue from the plant source: reddish brown fragments of cork, individual and groups of polyhedral to oblong stone cells, partly with greatly thickened, pitted, and lignified walls and brownish contents; fragments of thin-walled parenchyma and sclerenchymatous fibres, and irregular prismatic to polyhedral crystals of calcium oxalate.

Myrrha should be protected from light and moisture in well-closed containers. It is best with a desiccant, since the carbohydrate part of the drug readily absorbs water. Preferably, myrrha should not be stored in powdered form.

Myrrha has many medicinal benefits. Myrrha is a pungent, stimulating, astringent, carminative, astringent and aromatic herb. Myrrha also has anti-catarrhal, anti-inflammatory, antimicrobial, antiseptic, astringent, balsamic, carminative, cicatrisant, emmenagogue, expectorant, fungicidal, sedative, digestive, stomachic, tonic, and vulnerary properties.

Significantly, Myrrha can be used externally as a topical preparation to help treat infected wounds, minor skin inflammations, as well as inflammation of the throat, gums and mouth, including mouth ulcers, gingivitis, stomatitis and sinusitis. Myrrha can also help treat sores, such as: eczemas, skin ulcers and bedsores.

Myrrha can be helpful to relieve spasms, promote healing, fight inflammation, and reduce digestive discomfort. Myrrha also enhances cell regeneration.

Myrrh can be used internally to help treat stomach and chest problems, bronchial infection, and menstrual problems.

### Goldenseal

Goldenseal (Golden Seal) has a botanical name of Hydrastis Canadensis (LINN) and is from the family of N.O. Ranunculaceae. Golden Seal is also known as orange root, yellow root, BBR, berberine bisulfate, curcuma, eye balm, eye root, golden root, goldensiegel, goldsiegel, ground raspberry, guldsegl, hydrastidis rhizoma, hydrophyllum, Indian dye, Indian paint, Indian plant, Indian turmeric, jaundice root, kanadische gelbwurzel, kurkuma, Ohio curcuma, tumeric root, warnera, wild curcuma, wild turmeric, yellow eye, yellow Indian plant, yellow paint, yellow paint root, yellow puccoon, yellow seal, yellow wort, Indian turmeric, or curcuma.

Golden Seal is an herb that grows wild in moist mountainous woodland areas. The Golden Seal plant is a native of Ontario, Canada and the eastern United States, primarily, Ohio, Kentucky, West Virginia, Indiana, New York. Most of the commercial supplies are obtained from the Ohio Valley.

The generic name of Golden Seal is, Hydrastis, which is derived from two Greek words, signifying water and to accomplish, probably given it from its effect on the mucous membrane. Golden Seal belongs to the Buttercup family, Ranunculaceae, though its leaves and fruit somewhat resemble those of the Raspberry and the Rubus genus generally. Golden Seal is a small perennial herb, with a horizontal, irregularly knotted, bright yellow root-stock, from ¼ inch to ¾ inch thick, giving off slender roots below and marked with scars of the flower-stems of previous years. The flowering stem of Golden Seal, which is pushed up early in the spring, is from 6 to 12 inches high, erect, cylindrical, hairy, with downward-pointing hairs, especially above, surrounded at the base with a few short, brown scales. Golden Seal has two prominently-veined and wrinkled, dark green, hairy leaves, placed high up, the lower one stalked, the upper stalkless, roundish in outline, but palmately cut into 5 to 7 lobes, the margins irregularly and finely toothed. Golden Seal has one solitary radical leaf on a long foot-stalk, similar in form to the stem leaves, but larger, when full-grown being about 9 inches across. The flower of Golden Seal is produced in April and is solitary, terminal, erect, and small, with three small greenish-white sepals, falling away immediately after expansion, and has no petals and numerous stamens. The fruit of Golden Seal is a head of small, fleshy, oblong, crimson berries, tipped with the persistent styles and containing one or two hard black, shining seeds. Golden Seal is ripe in July and has much the appearance of a Raspberry (whence the name ‘Ground Raspberry’). The roots of Golden Seal are bright yellow

Numerous references to Golden Seal began to appear in medical writings as far back as 1820 as a strong tea for indigestion. Golden Seal has a long history of use among North Americans. Golden Seal flourished after the Civil War as it was an ingredient in many patent medicines.

The composition of Golden Seal comprises isoquinoline alkaloids, mainly hydrastine, berberine, berberastine, canadine, candaline, and hydrastinine; fatty acids, resin, polyphenolic acids, meconin, chlorogenic acid, phytosterins, and a small amount of volatile oil. The active ingredients in Golden Seal are the alkaloids hydrastine and berberine.

In particular, the primary constituents of Golden Seal (Hydrastis rhizome) are: the alkaloids burberine (3.5% to 4% by weight) which constitutes the yellow coloring matter of the drug, hydrastine (2% to 4% by weight), a crystallizable substance and a third alkaloid, Canadine; resin, albumin, starch, fatty matter, sugar, lignin and a small quantity of volatile oil, to which its odor is due, is also present. The rhizome can be much richer in alkaloid than the roots. Hydrastis owes its virtues almost entirely to hydrastine, the alkaloid berberine, apart from some effect as a bitter being practically inert. For many years the alkaloids and the powdered root were the primary forms administered of Golden Seal, but now the fluid extract is the form most used.

Golden Seal can be used as a wash for skin diseases, wounds, and for sore, inflamed eyes. Golden Seal can also be used to treat symptoms of the cold and flu and as an astringent, antibacterial remedy for the mucous membranes of the body. Golden Seal can further be used for treatment for the common cold and upper respiratory tract infections in combination Echinacea. Animal and laboratory research suggests that the Golden Seal (berberine) has effects against bacteria and inflammation. Berberine (Golden Seal) in addition to a standard prescription drug regimen can be used for chronic congestive heart failure (CHF) and are considered by some people to improve quality of life, and decrease ventricular premature complexes (VPCs) and mortality.

Golden Seal is sometimes suggested to be an immune system stimulant. Berberine (Golden Seal) can be used as a treatment for diarrhea caused by bacterial infections (including diarrhea from cholera). Golden Seal (berberine) can also be useful against bacteria and inflammation, as well in the eye to treat trachoma. Golden seal can further be hepatic, alternative, anti-catarrhal, anti-microbial, anti-inflammatory, astringent, laxative, expectorant, emmenagogue, and/or oxytocic. Golden Seal can also be used as a natural antibiotic and as a remedy for various gastric and genitourinary disorders.

Golden Seal can be used in many combination formulas and can enhance the potency of other herbs. Preparations of Golden Seal have been marketed for the treatment of menstrual disorders, urinary infections, rheumatic and muscular pain and as an antispasmodic.

Golden Seal can destroy many types of bacterial and viral infections. Golden Seal can also reduce gastric inflammation and relieve congestion. Berberine (Golden Seal) is a bitter that aids digestion and that has a sedative action on the central nervous system.

Golden Seal can be useful in combination with Echinacea particularly at the onset of cold and flu symptoms, especially coughs and sore throats. Golden Seal is an herb that can be helpful to strengthen the immune system, act as an antibiotic, have anti-inflammatory and antibacterial properties, potentiate insulin, and cleanse vital organs.

Golden Seal can be useful to promote the functioning capacity of the heart, the lymphatic and respiratory system, the liver, the spleen, the pancreas, and the colon. Taken internally, Golden Seal can be useful to increase digestive secretions, astringes the mucous membranes that line the gut, and check inflammation. Golden Seal can also help digestion by promoting the production of saliva, bile, and other digestive enzymes. Furthermore, Golden Seal can be useful to control heavy menstrual and postpartum bleeding by means of its astringent action. Moreover, Golden Seal can be a useful to help treat disorders of the stomach and intestines such as irritable bowel syndrome, colitis, ulcers, and gastritis and internal parasites.

As a dilute infusion, Golden Seal can be used as an eyewash and as a mouthwash for gum disease, and canker sores. Golden Seal can also an effective wash or douche for yeast infections. Golden Seal can further be helpful to stop diarrhea, as well as bleeding. Golden Seal can also stimulate appetite and also helps to aid digestion and the flow of gastric juices and bile. Moreover, Golden Seal acts as a mild laxative, while assisting with peptic ulcers. Golden Seal can be helpful women to help treat painful menstruation and pelvic inflammation disease.

Golden Seal can be a laxative, alternative and detergent. Golden Seal can be a valuable remedy in the disordered conditions of the digestion and has a special action on the mucous membrane, making it of value as a local remedy in various forms of catarrh. In chronic inflammation of the colon and rectum, injections of Hydrastine from Golden Seal are often helpful and have been used in hemorrhoids with excellent results. The alkaloid hydrastine in Golden Seal can have an astringent action. Golden Seal powder has proved useful as a snuff for nasal catarrh. Golden Seal can also be useful to treat dyspepsia and gastric catarrh, as well as loss of appetite and liver troubles. As a tonic, Golden Seal can be helpful to treat habitual constipation, given as a powder, combined with any aromatic. Furthermore, Golden Seal can be useful to help treat sickness due to vomiting.

Moreover, Golden Seal can be useful to help treat: abnormal heart rhythms, acne, AIDS, alcoholic liver disease, anal fissures, anesthetic, antibacterial, anticoagulant (blood “thinning”), antifungal, anti-heparin, antihistamine, anti-inflammatory, astrocytoma, atherosclerosis (“hardening” of the arteries), anxiety, appetite stimulant, arthritis, asthma, athlete's foot, bile flow stimulant, blood circulation stimulant, boils, bronchitis, cancer, candida yeast infections, canker sores, cervicitis, chemotherapy adjuvant, chicken pox, chronic fatigue syndrome, colitis (intestinal inflammation), conjunctivitis, constipation, Crohn's disease, croup, cystic fibrosis, cystitis, dandruff, deafness, diabetes mellitus, diarrhea, digestion problems, diphtheria, diuretic (increasing urine flow), eczema, enhancement of insulin effects, eyewash, fever, fistula problems, flatulence (gas), gallstones, gangrene, gastroenteritis, genital disorders, giardia infection, gingivitis, glioblastoma, headache, H. pylori infection, hemorrhage (bleeding), hemorrhoids, hepatitis, herpes, hiatal hernia, high blood pressure, high tyramine levels, hypoglycemia (low blood sugar levels), impetigo, inducing (causing) abortion, indigestion, infections, influenza, itching, jaundice, keratitis (inflammation of the cornea of the eye), leishmaniasis, liver disorders, lupus, menstruation problems, morning sickness, mouthwash, muscle pain, muscle spasm, night sweats, obesity, osteoporosis, otorrhea (discharge of fluid from the ear), pain, pneumonia, premenstrual syndrome, prostatitis, psoriasis, sciatica, seborrhea, sedative, sinusitis, stomach ulcers, stimulant, strep throat, syphilis, tetanus, thrombocytopenia (low blood platelets), thrush, tinnitus (ringing in the ears), tonsillitis, tooth disease, trichomoniasis, tuberculosis, urinary tract disorders, uterus inflammation, uterus stimulant, vaginal irritation, and varicose veins.

Golden Seal can be used to topically treat skin disorders such as psoriasis, eczema, athlete's foot, herpes, and ringworm, as well as to help treat muscle aches and pain. Golden Seal can also be useful as a medicinal preparation to topically treat eczema, ear inflammation, conjunctivitis, and vaginal inflammations, as well as gum disease.

### Cayenne

Cayenne is also known under the botanical name of Capsicum minimum (ROXB) with a family name of N.O, Solanaceae and is sometimes referred to as African Pepper. Chillies. Bird Pepper. Cayenne or Capsicum derives its name from the Greek, ‘to bite,’ in allusion to the hot pungent properties of the fruits and seeds.

Historically, Cayenne pepper was introduced into Britain from India in 1548, and Gerard mentioned it as being cultivated in his time. The Cayenne plant was described by Linnaeus under the name of C. frutescens proper. Cayenne is grown in Zanzibar and other places.

Cayenne is a shrubby perennial plant 2 to 6 feet high. Branches of Cayenne are angular, usually enlarged and slightly purple at the nodes. Cayenne has bell-shaped, white to green flowers which appear in spring and summer, and are followed by hollow fruits, which display differing colors when ripe. Cayenne has medium petioles, slender peduncles which are often in pairs, and longer than the fruit; a cup-shaped calyx, a clasping base of fruit which is red, ovate, and long; and flat small seeds which number from ten to twenty-nine. The cuticle of the pericarp of Cayenne is uniformly striated and in this particular is distinct from other species. The taste of Cayenne is very pungent and has a noticeable smell.

Cayenne pepper can be produced from the capsicum herb, which is a tender, variable annual with branched stems and simple oval, lance-shaped leaves. African Cayenne pepper is generally light brownish-yellow color and very pungent; its pungency appears to depend on a component Capsicin.

Cayenne can comprise a pepper with up to 1.5% capsaicinoids including 0.1%-1% capsaicin, 6,7-dihydrocapsaicin, nordihydrocapsaicin, homodihydrocapsaicin, and homocapsaicin; fixed oils; carotenoid pigments including capsanthin, capsorubin, alpha- and beta-carotene; steroid glycosides, including capsicosides A, B, C, and D; 9-17% fats; 12-15% proteins; vitamins A and C; and a trace of volatile oil. Generally, Cayenne can also comprise Capsaicin, a red coloring matter, oleic, palmitic and stearic acids.

Cayenne can be useful for ailments of the intestines and stomach. Cayenne can further be useful to reduce dilated blood-vessels and relieve chronic congestion Herbalists use it largely in pill form and powdered. Medicinally, Cayenne is particularly useful in treating various forms of topical pain such as arthritis, lumbago and spasms because when applied topically to the skin, the capsaicin in Cayenne causes a sensation of pain and warmth, which can cause extended reversible insensitivity of the skin. Cayenne can also be used externally for sprains, itching, arthritis, unbroken chilblains, and neuralgia. Externally Cayenne can be applied to the skin as a rubefacient and acts gently with little danger of blistering (vesication). Cayenne can be applied as a cataplasm, liniment, or medicinal preparation.

Cayenne can be used internally to improve circulation and to help treat fevers, varicose veins, asthma, and digestive and gastrointestinal problems such as dyspepsia, colic and flatulence. Moreover, Cayenne can be used as a condiment.

Cayenne can be useful for laryngitis as a gargle. Cayenne can also be used as a local stimulant, with no narcotic effect.

### Myrtle

The Myrtle (Myrtus) is a genus of one or two species of flowering plants in the family Myrtaceae, native to southern Europe and northern Africa. Myrtle are evergreen shrubs or small trees, growing to 5 m tall. The leaves of Myrtle are entire, 3-5 cm long, with a pleasantly fragrant essential oil. The star-like flowers of Myrtle have five petals and sepals, and an amazingly large number of stamens. Petals are usually white, with globose blue-black berries containing several seeds.

The Common Myrtle Myrtus communis, is widespread in the Mediterranean region and is also by far the most commonly cultivated. The other species, Saharan Myrtle M. nivellei, is restricted to the Tassili n'Ajjer mountains in southern Algeria and the Tibesti Mountains in Chad, where it occurs in small areas of sparse relict woodland near the centre of the Sahara Desert; it is listed as an endangered species.

Preferably, the medical composition excludes undesired Myrtle and its extracts, which can inhibit or retard the unexpected advantageous results of the novel medicinal composition.

### Centaurea

Centaurea (Cen-tau-ré-a) is a genus of about 350-500 species of herbaceous thistle-like flowering plants in the family Asteraceae, and is widely found in the United States and Canada. Common names for different species of Centaurea include star-thistle, cornflower, knapweed and bluet. Some species of Centaurea are cultivated as ornamental plants in gardens.

Species of the genus Centaurea include: Centaurea adpressa, Centaurea aggregata, Centaurea akamantis, Centaurea alpestris, Centaurea alpina, Centaurea americana (Plectocephalus americanus)—American Star-thistle, Centaurea appendicigera-endemic in Turkey, Centaurea argentea, Centaurea aspera—Rough Star-thistle, Centaurea atropurpurea, Centaurea babylonica, Centaurea behen, Centaurea bella, Centaurea bovina, Centaurea bulbosa, Centaurea cachinalensis, Centaurea calcitrapa—Purple Star-thistle or Caltrop—Europe, Centaurea calcitrapoides, Centaurea chilensis, Centaurea cineraria—Dusty Miller, Centaurea clementei, Centaurea cyanus—Cornflower, Centaurea dealbata, Centaurea debeauxii, Centaurea depressa, Centaurea diffusa—Diffuse Knapweed, Centaurea diluta, Centaurea dschungarica, Centaurea eriophora, Centaurea floccosa, Centaurea gayana, Centaurea glastifolia, Centaurea grinensis, Centaurea gymnocarpa, Centaurea horrida, Centaurea hypoleuca, Centaurea iberica—A spiny Mediterranean species thought to be the “thistle” mentioned in Genesis, Centaurea imperialis, Centaurea jacea, Centaurea kasakorum, Centaurea kopetaghensis, Centaurea kotschyana, Centaurea leucophylla, Centaurea macrocephala, Centaurea maculosa—Spotted Knapweed; eastern Europe; introduced in North America, now an invasive weed which releases a toxin that reduces growth of forage species, Centaurea marschalliana, Centaurea melitensis, Centaurea moschata—Sweet Sultan, Centaurea monocephala, Centaurea montana—Perennial Cornflower, Centaurea nigra—Black Knapweed or Common Knapweed, Centaurea nigrescens—Tyrol Knapweed; southern and eastern Europe, Centaurea orientalis, Centaurea ovina, Centaurea paniculata, Centaurea phrygia, Centaurea pindicola, Centaurea polypodiifolia, Centaurea pulcherrima, Centaurea ragusina, Centaurea rothrockii (Plectocephalus rothrockii), Centaurea ruthenica, Centaurea rutifolia, Centaurea sadleriana—Pannonian Knapweed, Centaurea scabiosa—Greater Knapweed, Centaurea seridis, Centaurea sibirica, Centaurea simplicicaulis, Centaurea solstitialis—Yellow Star-thistle; Europe, Centaurea squarrosa, Centaurea stenolepis, Centaurea stoebe, Centaurea sulphurea, Centaurea transalpina, Centaurea tchihatcheffii—, Mogan Lake, Ankara, Turkey, Centaurea trichocephala, Centaurea triniifolia, Centaurea triumfettii, Centaurea uniflora, and Centaurea virgata.

Preferably, the medical composition excludes undesired Centaurea and its extracts, which can inhibit or retard the unexpected advantageous results of the novel medicinal composition.

### Menthol

Menthol is an organic compound made synthetically or obtained from peppermint or other mint oils. Menthol is a waxy, crystalline substance, clear or white in color, which is solid at room temperature and melts slightly above. The main form of Menthol occurring in nature is (−)-Menthol, which is assigned the (1R,2S,5R) configuration. Mentha arvensis is the primary species of mint used to make natural Menthol crystals and natural Menthol flakes. This species is primarily grown in the Uttar Pradesh region in India.

Menthol has a molecular formula of C₁₀H₂₀O and a chemical name of 2-(2-Propyl)-5-methylcyclohexanol, and is also sometimes knows as 3-p-Menthanol, Hexahydrothymol, Menthomenthol, or peppermint camphor. The chemical formula of Menthol is shown hereinafter.

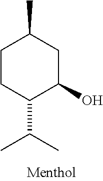

Natural Menthol exists as a pure enantiomer, nearly always the (1R,2S, SR) form (bottom left of diagram below). The other seven stereoisomers of Menthol are:

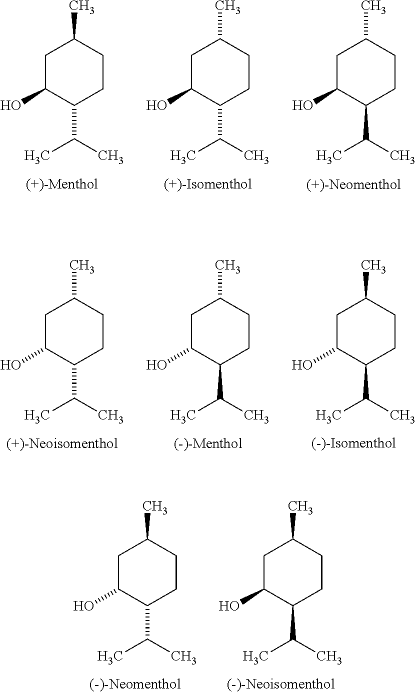

In the natural compound of Menthol, the isopropyl group is oriented trans-orientation to both the methyl and alcohol groups. Menthol can be drawn in any of the ways shown hereinafter:

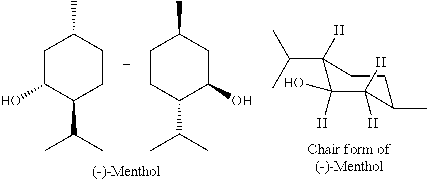

In the ground state of Menthol, all three bulky groups in the chair are equatorial, making (−)-Menthol and its enantiomer the most stable two isomers out of the eight.

Menthol is a white or colorless crystalline solid with a boiling point of 212° C. (485 K). There are two crystal forms for racemic Menthol. Menthol has a melting point of 36-38° C. (311 K), racemic 42-45° C. (318 K), (−)-form (α) 35-33-31° C., (−)-isomer. Pure (−)-menthol has four crystal forms, of which the most stable is the α form.

Menthol reacts in many ways like a normal secondary alcohol. Menthol is oxidized to menthone by oxidizing agents such as chromic acid or dichromate, although under some conditions the oxidation can go further and break open the ring. Menthol is easily dehydrated to give mainly 3-menthene, by the action of 2% sulfuric acid. PCl₅ gives menthyl chloride.

The demand for Menthol greatly exceeds the supply from natural sources. Menthol is manufactured as a single enantiomer (94% ee) by Takasago International Co. on a scale of 400,000 tons per year. The process involves an asymmetric synthesis developed by a team led by Ryoji Noyori: The process of synthesizing Menthol begins by forming an allylic amine from myrcene, which undergoes asymmetric isomerisation in the presence of a BINAP rhodium complex to produce after hydrolysis enantiomerically pure R-citronellal. This is cyclised by a carbonyl-ene-reaction initiated by zinc bromide to isopulegol which is then hydrogenated to give pure (1R,2S, 5R)-Menthol.

Racemic Menthol can be prepared simply by hydrogenation of thymol. Menthol is also formed by hydrogenation of pulegone.

The ability of Menthol to chemically trigger cold-sensitive receptors in the skin provides the well known cooling sensation that it provokes when inhaled, eaten, or applied to the skin. Menthol does not cause an actual drop in temperature. Menthol has local anesthetic and counterirritant qualities, and it is widely used to relieve minor throat irritation.

Because of its cooling effect, Menthol is used in products meant to relieve skin irritation, sore throat, or nasal congestion. Menthol can be used to treat sunburn, fever, or muscle aches as well. In traditional Asian medicine, Menthol can be prescribed for nausea, diarrhea, indigestion, headache, cold, or sore throat. When used as a supplement for health reasons, Menthol is usually taken in the form of peppermint oil. Products that commonly contain menthol include toothpaste, cough drops, lip balm, mouthwash, gum, and cigarettes.

Menthol can be contained in non-prescription products for short-term relief of minor sore throat and minor mouth or throat irritation, for example in lip balms and cough medicines. Menthol is classed as an antipruritic, which reduces itching. Menthol can also be contained in combination products used for relief of muscle aches, sprains, and other conditions, as well as in decongestants. Menthol can be used as an ingredient in mouthwash. Menthol has also been used as an additive in various cigarette brands, both for flavor and to reduce the throat and sinus irritation caused by smoking.

Menthol can have many medicinal uses:
1. In cough medicines, for short-term relief of minor sore throats and minor mouth or throat irritation.
2. Lip balms.
3. Aantipruritic preparation to reduce itching.
4. As a topical analgesic to relieve minor aches and pains such as muscle cramps, sprains, headaches and similar conditions, alone or combined with other chemicals or products.
5. Gels and creams.
6. Patches.
7. Decongestants for chests and sinuses.
8. Nose inhaler.
9. Sunburn ointments and preparations.
10. Oral products and bad-breath remedies, such as for mouthwash, and toothpaste, mouth.
11. Food flavor agent; such as in chewing-gum and candy, as well as in soda, syrups, and water.
12. A pesticide against tracheal mites of honeybees.
13. Beauty products such as hair-conditioners.
14. To help treat indigestion, nausea, diarrhea, colds, and headaches.

### Benzalkonium Chloride

A surfactant can provide a broad spectrum of antimicrobial action. Surfactants can comprise quaternary ammonium salts containing 6-18 carbon atoms. Preferably, the quaternary ammonium salt surfactant is a mixture of alkyl dimethylbenzylammonium chlorides, which can comprise benzalkonium halide comprising benzalkonium bromide, benzalkonium fluoride and/or most preferably benzalkonium chloride.

The chemical formula of benzalkonium chloride is shown hereinafter. Other types of benzalkonium chloride can be used.

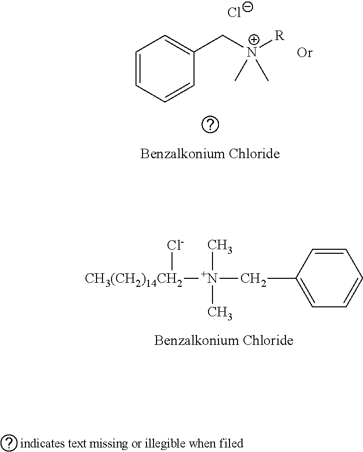

Benzalkonium chloride in aqueous solution is commercially available, among others, under the brand name and trade mark Zephiran® distributed by Sanofi Winthrop Pharmaceuticals (formerly Winthrop Labs).

Benzalkonium chloride can be obtained by extraction, such as a surfactant-mediated solid phase extraction procedure to obtain a concentration of benzalkonium (BA) chloride. For example, dodecyl sulfate attached to a strong anion exchange resin and aqueous samples are passed through a column containing this surfactant-resin material. Benzalkonium chloride can be adsorbed from the aqueous solution onto the sorbent via hydrophobic and electrostatic interactions. Electrostatic attractions occur between the benzalkonium cations and removable dodecyl sulfate anions. Removing this ion pair from the sorbent can result in efficient elutions.

Benzalkonium chloride (alkyl dimethyl benzyl ammonium chloride) is a mixture of alkylbenzyl dimethylammonium chlorides of various alkyl chain lengths. Benzalkonium chloride is a nitrogenous cationic surface-acting agent belonging to the quaternary ammonium group. Benzalkonium chloride has three main categories of use; as a biocide, a cationic surfactant and phase transfer agent in the chemical industry

Benzalkonium chloride is a rapidly acting anti-infective surfactant with a moderately long duration of action. The surfactant is active against bacteria and some viruses, fungi and protozoa. Bacterial spores are considered to be resistant. Solutions of benzalkonium chloride are bacteriostatic or bactericidal according to concentration. The exact mechanism of bacterial action of benzalkonium chloride is unknown but is thought to be due to enzyme inactivation. Activity of benzalkonium chloride generally increases with increasing temperature and pH. Gram-positive bacteria are more susceptible to benzalkonium chloride than gram-negative bacteria. Unfortunately, benzalkonium chloride is inactivated by soaps, anionic detergents, serum, and certain proteins. Benzalkonium chloride has fallen out of favor in some laboratories for the above reasons.

Benzalkonium chloride can be readily soluble in alcohol, and acetone. Dissolution of benzalkonium chloride in water is slow, and aqueous solutions of benzalkonium chloride are preferred, as it is easier to handle. Solutions of benzalkonium chloride can be neutral to slightly alkaline with colors ranging from clear to a pale yellow. Solutions of benzalkonium chloride tend to foam when shaken, have a bitter taste, and a faint almond-like odor in concentrated solutions.

Standard concentrates of benzalkonium chloride can be manufactured as 50% and 80% w/w solutions, and sold under trade names or brand names, such as BC50, BC80, BAC50, BAC80, etc. The 50% solution of benzalkonium chloride is purely aqueous, while more concentrated solutions require rheology modifiers (alcohols, polyethylene glycols, etc.) to minimize increases in viscosity or gel formation under low temperature conditions.

Use of benzalkonium chloride range from disinfectant formulations to microbial corrosion inhibition in the oilfield sector. Benzalkonium chloride has been considered one of the safest synthetic biocides known and has a long history of efficacious use. Benzalkonium chloride is useful for antiseptics, hygienic towelettes, and wet wipes.

Alcohol-free benzalkonium chloride solutions can be used for skin disinfection prior to withdrawing blood. Benzalkonium chloride can be used as a preservative and in cosmetics such as eye and nasal drops.

The greatest biocidal activity of benzalkonium chloride is associated with the C12-C14 alkyl derivatives. The mechanism of bactericidal and microbicidal action is thought to be due to disruption of intermolecular interactions. This can cause dissociation of cellular membrane bilayers, which can compromise cellular permeability controls and induces leakage of cellular contents. Other biomolecular complexes of benzalkonium chloride within the bacterial cell can also undergo dissociation.

Enzymes, which finely control a plethora of respiratory and metabolic cellular activities, are particularly susceptible to deactivation by benzalkonium chloride. Furthermore, intermolecular interactions and tertiary structures in such highly specific biochemical systems can be readily disrupted by cationic surfactants, such as benzalkonium chloride.

Benzalkonium chloride solutions can provide rapidly acting biocidal agents with a moderately long duration of action. Benzalkonium chloride is active against bacteria and some viruses, fungi, and protozoa.

Formulations using benzalkonium chloride blended with various quaternary ammonium derivatives can be used to extend the biodcidal spectrum and enhance the efficacy of benzalkonium chloride based disinfection products. This technique has been used to improve virucidal activity of quaternary ammonium-based formulations to healthcare infection hazards such as hepatitis, herpes, HIV, etc. Benzalkonium chloride solutions for hospital use tend to be neutral to alkaline, non-corrosive on metal surfaces, non-staining and safe to use on all washable surfaces.

### Diluents

### Sterile Water and Isopropyl Alcohol

The medicinal composition can be diluted with a diluent, such a water, preferably sterile water, and/or isopropyl alcohol (C₃H₇OH). Isopropyl alcohol forms an azeotrope with water at 87.4% alcohol. Isopropyl alcohol is soluble or miscible with water, ethyl ether, and ethyl alcohol.

Isopropyl alcohol is a colorless, flammable chemical compound with a strong odor. Isopropyl alcohol is also known as isopropanol, rubbing alcohol, the abbreviation IPA propan-2-ol, 2-Propanol; sec-Propyl Alcohol; Alcojel; Alcosolve 2; Avantin; Avantine; Combi-Schutz; Dimethylcarbinol; Hartosol; Imsol A; Isohol; Isopropanol; Lutosol; Petrohol; Propol; PRO; Takineocol; 1-Methylethyl Alcohol; iso-C₃H₇₀H; 2-Hydroxypropane; Propane, 2-hydroxy-; sec-Propanol; Propan-2-ol; i-Propylalkohol; Alcohol, rubbing; Alcolo; Alcool isopropilico; Alcool isopropylique; Alkolave; Arquad DMCB; iso-Propylalkohol; Isopropyl alcohol; Lavacol; Visco 1152; Alcosolve; Chromal′; i-Propanol; 2-Propyl alcohol; Spectrar; Sterisol hand disinfectant; UN 1219; (−)-2,3-O-Isopropyl alcohol; Alcohol; Alcowipe; DuPont brand zonyl FSA fluorinated surfactants; DuPont brand zonyl FSJ fluorinated surfactants; DuPont brand zonyl FSN fluorinated surfactants; DuPont brand zonyl FSP fluorinated surfactants; I.P.S.; n-Propan-2-ol; Rubbing alcohol; Sec-propyl; 1-methylethanol; and Propanol-2.

Rubbing alcohol, U.S.P./B.P. is a liquid prepared for topical application. Rubbing alcohol can be prepared from specially denatured alcohol and can contain 68.5%-71.5% volume/volume (vol/vol) (v/v) of absolute (i.e. 100%) alcohol. Individual manufacturers, however, often use their own formulation standards or specifications in which the ethanol content usually ranges from 70-95% v/v.

The British Pharmacopoeia and United States Pharmacopoeia define the term alcohol as a mixture of ethanol (Ethyl Alcohol, C₂H₅OH=46.07) and water of fixed proportions. Alcohol, B.P. is 96% ethanol v/v, and Alcohol, U.S.P. is 95% ethanol v/v. Therefore, in Great Britain, the term rubbing alcohol also refers to a mixture using ethyl alcohol.

In the United States, rubbing alcohol, U.S.P. and all preparations coming under the classification of rubbing alcohols must be manufactured in accordance with the requirements of the US Treasury Department, Bureau of Alcohol, Tobacco, and Firearms, using Formula 23-H (8 parts by volume of acetone, 1.5 parts by volume of methyl isobutyl ketone, and 100 parts by volume of ethyl alcohol). Rubbing alcohol contains 68.5-71.5% by volume of absolute ethyl alcohol, the remainder comprising water and the denaturants, with or without color additives, and perfume oils. Rubbing alcohol contains in each 100 mL not less than 355 mg of sucrose octaacetate or not less than 1.40 mg of denatonium benzoate. Rubbing alcohol also exists on pharmacy store shelves without the U.S.P. standard designation. The concentration of the ethyl alcohol component can vary but it is generally printed under the ingredient list of that particular product. A common concentration of ethyl alcohol in rubbing alcohol is 95%.

Isopropyl alcohol can be used as a disinfectant. Isopropyl alcohol or rubbing alcohol is widely used as an antiseptic for sterilizing surfaces or clean minor cuts or abrasions. Rubbing alcohol can be applied externally as a cooling, soothing application for bedridden patients and athletes. Rubbing alcohol can also be used for cleansing the surgeon's hands and instruments and for disinfection of the patient's skin prior to penetration of the skin by a hypodermic needle. As an antiseptic, rubbing alcohol can be good against vegetative bacteria and fair against fungi and viruses. Rubbing alcohol can cool and soothe skin. Isopropyl rubbing alcohol can also be supplied in a 99% concentration, such as for use to harden skin such as in the case of the feet of novice hikers. Sterilizing pads typically contain a 60-70% solution of isopropyl alcohol in water. Isopropyl alcohol can be used as a disinfectant.

The composition can comprise a topical analgesic, pharmaceutical product, medicinal preparation, or medicine. The composition can also comprise a veterinary medicine or veterinary medicinal composition for treating, alleviating, or relieving conditions or symptoms associated with viral or bacterial infections or infectious diseases in dogs, cats, birds, horses, cows, sheep, swine (pigs or hogs), or other farm animals, rodents, or animals in zoos. In some circumstances, it may be useful to use the novel composition systemically.

The topical analgesic can have therapeutic benefits for use in humans, equine, bovine, canine, feline, porcine, or other animals or birds for treatment and healing of ligaments, tendons, muscles, bones, hematomas, nerves, or sports injuries or for epidermal or dermal treatment of arthritis, neuralgia, or pain; as well as for topical treatment for minor wounds and injuries.

The pharmaceutical product may also comprise a pain reliever or a muscle relaxant. Furthermore, the improved medicinal composition and treatment can be useful to help alleviate wounds, bruises, cold sores, and sores, rashes and skin conditions in people (human beings) and animals.

Among the many advantages of the medicinal composition are:
1. Superb treatment, healing or relief of symptoms resulting from injured ligaments, tendons, muscles, bones, hematomas, nerves, arthritis, or neuralgia.
2. Superior results in minimizing and relieving pain.
3. Excellent relief or treatment of skin conditions associated with various ailments.
4. Outstanding performance.
5. Comfortable.
6. Reliable.
7. Helpful.
8. Convenient.
9. Non-abrasive.
10. Attractive.
11. Easy to use.
12. Beneficial.
13. Smooth.
14. Soft.
15. Safe.
16. Economical.
17. Efficient.
18. Effective.
19. Soothing.
20. Fast-working.

Although embodiments of the invention have been shown and described, it is to be understood that various modifications and substitutions, different materials, and rearrangements or different proportions of compounds, herbal extracts, plants, ingredients, parts, or components, as well different method steps, and additional uses and treatments can be made by those skilled in the art without departing from the novel spirit and scope of this invention.

What is claimed is:

1. A medicinal composition, comprising:
a first plant group consisting of primary plants;
said primary plants consisting of extracts or portions of primary herbaceous botanicals selected from the group consisting of the genus of Bellis Perennis (Daisy), Ruta Graveolens (Rue), Comfrey, Elder, and Hops;
said primary herbaceous botanicals consisting of at least two (2) different primary herbaceous botanicals and their extracts;
said primary herbaceous botanicals being present in said medicinal composition in the absence of Myrtle and Centaurea; and
said primary herbaceous botanicals cooperating with each other in said medicinal composition to help treat ailments or diseases.

2. A medicinal composition in accordance with claim 1 wherein said primary herbaceous botanicals consist of at least three (3) different primary herbaceous botanicals.

3. A medicinal composition in accordance with claim 1 wherein said primary herbaceous botanicals consist of at least four (4) different primary herbaceous botanicals.

4. A medicinal composition in accordance with claim 1 wherein said primary herbaceous botanicals consist of five (5) different primary herbaceous botanicals.

5. A medicinal composition in accordance with claim 1 including:
a second plant group consisting of secondary plants;
said secondary plants consisting of extracts or portions of secondary herbaceous botanicals selected from the group consisting of the genus of Echinacea, Hypericum Perforatum (St. John's Wart), Aloe, and Mistletoe;
said Aloe comprising extracts or portions of an Aloe plant selected from the group consisting of Aloe Barbadensis, Aloe Capensis, and Aloe Vera;
said secondary herbaceous botanicals consisting of at least one (1) secondary herbaceous botanical;
said secondary herbaceous botanicals being present in said medicinal composition in the absence of Myrtle and Centaurea and their extracts; and
said primary and secondary herbaceous botanicals cooperating with each other in said medicinal composition to help treat ailments or diseases.

6. A medicinal composition in accordance with claim 1 wherein said secondary herbaceous botanicals consist of at least two (2) different secondary herbaceous botanicals.

7. A medicinal composition in accordance with claim 1 wherein said secondary herbaceous botanicals consist of at least three (3) different secondary herbaceous botanicals.

8. A medicinal composition in accordance with claim 1 wherein said secondary herbaceous botanicals consist of at least four (4) different secondary herbaceous botanicals.

9. A medicinal composition in accordance with claim 1 including:
a third plant group consisting of tertiary plants;
said tertiary plants consisting of extracts or portions of 1-6 different tertiary herbaceous botanicals selected from the group consisting of the genus of Rhus toxicodendron (Poison Ivy), Commiphora myrrha, Goldenseal, Cayenne, Eucalyptus, and Camphor;
said tertiary herbaceous botanicals being present in said medicinal composition in the absence of Myrtle and Centaurea and their extracts and
said primary, secondary and tertiary herbaceous botanicals cooperating with each other in said medicinal composition to help treat ailments or diseases.

10. A medicinal composition in accordance with claim 1 further comprising water and/or Isopropyl Alcohol.

11. A medicinal composition in accordance with claim 1 further comprising Benzalkonium halide selected from the group consisting Benzalkonium chloride, Benzalkonium bromide, and Benzalkonium fluoride.

12. A medicinal composition in accordance with claim 1 comprising:
said primary herbaceous botanicals consist of 2-5 different primary herbaceous botanicals;
said secondary herbaceous botanicals consist of 1-4 different secondary herbaceous botanicals;
said tertiary herbaceous botanicals consist of 1-6 different tertiary herbaceous botanicals;
Benzalkonium chloride; and
at least one compound comprising Menthol or a diluent comprising distilled water and/or Isopropyl Alcohol; and
said primary, secondary and tertiary herbal extracts cooperating with each other and said Benzalkonium chloride and said Menthol or said diluent in said medicinal composition to help treat ailments or diseases; and
said composition further comprising a topical analgesic with therapeutic benefits for use in humans, equine, bovine, canine, feline, porcine, or other animals or birds for treatment and healing of ligaments, tendons, muscles, bones, hematomas, nerves, or sports injuries or for epidermal or dermal treatment of arthritis, neuralgia, or pain.

13. A medicinal composition, comprising:
primary herbal extracts of at least a portion of five (5) different primary plants consisting of primary herbaceous botanicals comprising by weight based upon the total weight of the composition:
from about 0.01% to about 90% Bellis Perennis (Daisy);
from about 0.003% to about 92% Ruta Graveolens (Rue);
from about 0.001% to about 92% Comfrey,
from about 0.003% to about 94% Elder; and
from about 0.001% to about 88% Hops; and
said primary herbal extracts of said primary herbaceous botanicals cooperating with each other in said medicinal composition to help treat ailments or diseases.

14. A medicinal composition in accordance with claim 13 further comprising:
secondary herbal extracts of at least a portion of three (3) different secondary plants consisting of secondary herbaceous botanicals comprising by weight based upon the total weight of the composition:
from about 0.01% to about 90% Echinacea purpurea;
from about 0.005% to about 98% Hypericum Perforatum (St. John's Wart);
from about 0.002% to about 95% Aloe Vera;
from about 0% to about 84% Mistletoe; and
said primary and secondary herbal extracts cooperating with each other in said medicinal composition to help treat ailments or diseases.

15. A medicinal composition in accordance with claim 14 wherein said Echinacea purpurea comprises by weight based upon the total weight of the composition:
from about 0.3% to about 9% by weight echinacoside;
from about 0.1% to about 7% by weight PS I (4-O-methyglucoronoarabinoxylan, M.sub.r 35 kD) and PS II (acid rhamnoarabinogalactan, M.sub.r 450 kD);
from about 0.1% to about 10% by weight cynarin (1,5-di-O-caffeoylquinic acid) and chicoric acid (2,3-O-di-caffeoyltartaric acid);
from about 0.2% to about 4% by weight echinolone;
from about 0.2% to about 8% by weight echinacin B;
from about 0.1% to about 6% by weight echinaceine;
from about 2% to about 7% by weight anthonocyanins comprising cyanidin 3-O-B-D-glucopyranoside and 3-O-(6-O-malonyl-B-D-glucopyranoside);
from about 0.01% to about 0.06% by weight pyrrolizidine alkaloids comprising tussilagine and isotussilagine;
from about 0.003% to about 0.009% by weight isomeric dodeca isobutylamides and tetroenoic acid; and
from about 0.01% to about 2% by weight carophylenes.

16. A medicinal composition in accordance with claim 14 further comprising:
tertiary herbal extracts of at least a portion of six (6) different tertiary plants consisting of tertiary herbaceous botanicals comprising by weight based upon the total weight of the composition:
from about 0.001% to about 98% Rhus toxicodendron (Poison Ivy);
from about 0.004% to about 86% Commiphora myrrha;
from about 0.002% to about 83% Goldenseal;
from about 0.5% to about 75% Cayenne;
from about 0.1% to about 10% Eucalyptus;
from about 0.1% to about 10% Camphor;
said primary, secondary and tertiary herbal extracts cooperating with each other in said medicinal composition to help treat ailments or diseases.

17. A medicinal composition in accordance with claim 16 further comprising by weight based on the total weight of the composition:
from about 0.03% to about 27% Benzalkonium chloride; and
from about 0.02% to about 98% Isopropyl Alcohol; and
said Isopropyl Alcohol and said Benzalkonium chloride cooperating with said primary, secondary and tertiary herbal extracts in said medicinal composition to help treat ailments or diseases.

18. A medicinal composition in accordance with claim 17 further comprising by weight based on the total weight of the composition:
from about 40% to about 60% water; and
said water cooperating with said Isopropyl Alcohol, Benzalkonium chloride and said primary, secondary and tertiary herbal extracts in said medicinal composition to help treat ailments or diseases.

19. A medicinal composition in accordance with claim 13, comprising by weight based upon the total weight of the medicinal composition:
said primary herbal extracts consisting of
from about 4% to about 8% Bellis Perennis (Daisy);
from about 4% to about 8% Ruta Graveolens (Rue);
from about 4% to about 8% Comfrey,
from about 4% to about 8% Elder; and
from about 4% to about 8% Hops;
said secondary herbal extracts consisting of
from about 2% to about 4% Echinacea purpurea;
from about 2% to about 4% Hypericum Perforatum (St. John's Wart);
from about 2% to about 4% Aloe Vera;
from about 0% to about 4% Mistletoe;
said tertiary herbal extracts consisting of
from about 0.5% to about 1.5% Rhus toxicodendron (Poison Ivy);
from about 0.5% to about 1.5% Commiphora myrrha;
from about 0.5% to about 1.5% Goldenseal; and
from about 0.5% to about 1.5% Cayenne;
from about 1% to about 4% Eucalyptus;
from about 1% to about 4% Camphor;
from about 0% to about 4% Menthol;
from about 0.05% to about 0.2% Benzalkonium chloride;
from about 60% to about 80% Isopropyl Alcohol; and
from about 40% to about 60% distilled water; and
said primary, secondary, and tertiary herbal extracts of said herbaceous botanicals cooperating with each other and said Menthol, Isopropyl Alcohol, Benzalkonium chloride and/or said distilled water in said medicinal composition to help treat ailments or diseases;
said composition comprising a topical analgesic, pharmaceutical product, medicinal preparation, or medicine; and
said topical analgesic having therapeutic benefits for use in humans, equine, bovine, canine, feline, porcine, or other animals or birds for treatment healing, or relief of symptoms resulting from injured ligaments, tendons, muscles, bones, hematomas, nerves, or sports injuries or for epidermal or dermal treatment, healing, or relief of symptoms resulting from arthritis, neuralgia, or pain.
